(19) United States
(12) Patent Application Publication
(10) Pub. No.: US 2012/0263858 A1
(43) Pub. Date: Oct. 18, 2012

(54) ISOLATED PLANT GDSL LIPASE

(76) Inventors: Bénédicte Bakan, Nantes (FR); Christophe Rothan, Saint Selve (FR); Martine Chamley, Leognan (FR); Didier Marion, Nantes (FR); Marie-Laure Girard, Brian Sur L'Authion (FR); Fabien Mounet, Pineuilh (FR)

(21) Appl. No.: 13/517,238

(22) PCT Filed: Dec. 21, 2010
(86) PCT No.: PCT/EP2010/070384

§ 371 Date: Jun. 19, 2012

Related U.S. Application Data
(60) Provisional application No. 61/288,464, filed on Dec. 21, 2009.

(51) Int. Cl. C12N 9/20; C12N 1/21; C12N 1/15; C12N 1/19; C12N 5/10; A23L 1/28; C12N 15/55; C07K 16/40; A01H 5/00; A01H 5/08; A23L 1/212; A23L 2/02; C12N 15/63; C40B 20/08

(52) U.S. Cl. 426/599; 435/198; 435/320.1; 435/252.3; 435/254.11; 435/254.2; 435/419; 435/252.33; 506/6; 536/23.2; 530/387.9; 800/298; 800/317.4; 426/615; 426/655

(57) ABSTRACT

The invention relates to an isolated plant GDSL-lipase polypeptide differentially expressed between the different steps of the plant development and/or the different tissues of the plant, a nucleic acid molecule encoding it, and a plant or part of plant genetically engineered to not express it at least partially.

Described herein are inventions in the field of genetic engineering of plants, including isolated nucleic acid molecules encoding polypeptides to improve agronomic, horticultural, and quality traits. This invention relates generally to nucleic acid sequences encoding proteins that are related to the cutin polymerization in plants. More specifically, the present invention relates to nucleic acid sequences encoding polypeptides being capable of altering the cutin and the use of these sequences in transgenic plants. In particular, the invention is directed to methods for manipulating cuticle polymerization in plants and fruits.

Plant cuticle is a hydrophobic layer that covers all aerial surfaces. Cuticle is a complex hydrophobic assembly characterized by a biopolymer, cutin, coated and filled by waxes. Waxes display a high chemical diversity in the plant kingdom and generally consist of mixtures of aliphatic molecules with very long carbon-chain (up to C60) including alkanes, primary and secondary alcohols, aldehydes, fatty acids, esters as well as cyclic triterpenoids, phenylpropanoids and phenolics (Waltson, 1990).

Cutin, the major component of cuticle, is a polyester of ω and mid-chain hydroxyl C16 and C18 fatty acids and glycerol (Kolattuduky, 1996; Graça et al., 2002). Cutin fulfils multiple functions in plants such as control of non stomatal water loss (Sieber et al., 2000), permeation of gases and solutes (Kersteins, 1996; Riederer and Schreiber, 2001).

Cutin plays also an essential role in the regulation of cell adhesion during plant development by preventing organ fusion as observed in Arabidopsis mutants (Sieber et al., 2000; Nawrath, 2006) or by participating to hull adhesion in grains (Takeda et al., 2008). It is generally admitted that plant cuticles and its polymeric matrix, cutin, are primary barriers to pathogens and that cutin monomers released by fungal cutinase are signalling molecules for both pathogen and plants (Gilbert et al., 1996; Schweizer et al., 1996; Iwamoto et al., 2002).

Cutin monomers, i.e. hydroxy- and epoxy-fatty acids, are synthesized in the epidermal cells from which they are exported and polymerized at the surface of the cells.

Several genes encoding enzymes involved in the biosynthesis of cutin monomers have been identified through Arabidopsis fusion mutants (Wellesen et al., 2001; Schnurr et al., 2004; Xiao et al., 2004; Bessire et al., 2007; Kannangara et al., 2007). The discovery of a transcription factor WIN1 that regulates different genes of cutin biosynthesis has provided a model pathway. This major pathway involved ω-hydroxylases, long-chain acyl-coenzyme A synthetases and glycerol-3-phosphate acyltransferases (Kannangara et al., 2007).

Another pathway, the lipoxygenase-peroxygenase pathway, could be involved in the midchain epoxidation and, coupled with epoxyhydrolases, in the mid-chain hydroxylation of long chain w-hydroxylated fatty acid precursors (Blee and Schuber, 1993). Glycerol-3-phosphate acyltransferases (GPAT), catalyse the transfer of acyl-CoAs to glycerol-3-phosphate to form lysophosphatidic acid a precursor of acylglycerols. GPAT are also involved in the formation of suberin, a cutin-like polymer that accumulates in roots and in stems (Beisson et al., 2007) and in cutin formation (Li et al., 2007). Therefore monoacylglycerols and lysophosphatidic acids could be candidate precursors for cutin polymerization after translocation in the apoplastic compartment (Li et al., 2007; Pollard et al., 2008). In this regard, it has been demonstrated that, as observed for wax monomers, ATP-binding cassette (ABC) transporters located in the plasmalemma are essential for the exportation of cutin precursors through the plasma membrane from cell into the apoplast (Panikashvili et al., 2007; Bird, 2008).

By contrast, the mechanisms involved in the transport of cutin precursors through the apoplast and its polymerisation are less documented. Acceptor is needed to create lipophilic environment in order to diffuse through the aqueous hydrophilic apoplast and reach the surface polymerisation site. Due to their apoplastic localisation and to their ability to bind lipids in their hydrophobic cavity, LTPs have been often suggested for this function (Sterk et al., 1991; Pyee et al., 1994; Suh et al., 2005). However, no experimental data has confirmed so far the transport function of plant LTPs. Concerning polymerisation of the cutin precursors, two mechanisms can be actually considered, i.e. a chemical or an enzymatic transesterification process. Chemical trans-esterification could be catalysed by the slightly acidic pH of the apoplastic compartment, but is, in a first attempt, a mechanism too slow to respond at the rapid growth of cuticle during plant development.

Actually, two enzymes are good candidates to perform the polymerization of cutin precursors. First of all, BODYGUARD, an epidermis-specific and extracellular α/β hydrolase-protein whose interruption in Arabidopsis induces deformation of epidermal cell walls and an irregular deposition of cutin. Furthermore, the mutant accumulates more cutin and wax monomers than the wild plants (Kurdyukov et al., 2006). BDG could either play a role of cutin syntethase or participate to the control of cell proliferation and differentiation of the epidermis (Kurdyukov et al., 2006). In this later hypothesis, the modification of the epidermis cells would limit the formation of the cuticle layer (Kurdyukov et al., 2006; Pollard et al., 2008).

The second candidates are proteins of the GDSL family of lipase-hydrolases. These enzymes are located in the cell walls close to the cuticular membranes (Reina et al., 2007) and their expression is under the control of WIN1, the transcription factor that regulates cutin deposition (Kannangara et al., 2007).

However, except a role in tolerance to biotic and abiotic stress (Oh et al., 2005; Naranjo et al., 2006; Hong et al., 2008) no experiment has been conducted to relate these proteins with building of cuticular barriers. Although a transferase activity was demonstrated for microbial protein homologue so that this family is also called the GDSL108 motif lipase-acylhydrolase family (Akoh et al., 2004), only their hydrolytic activities towards lipid ester analogues were tested (Oh et al., 2005; Hong et al., 2008).

### SUMMARY OF THE INVENTION

This invention is based on the isolation and characterization of GDSL1 the main GDSL-motif lipase-acylhydrolase protein expressed in the exocarp of developing tomato fruit (Solanum lycopersicum var). Silencing of this protein leads to major alteration in the formation of cutin and an increase of the permeability of fruit cuticle.

This invention is also based on the characterization of GDSL3 expressed in the seeds of developing tomato fruit (Solanum lycopersicum var) and GDSL2 expressed in all tomato fruit tissues but only at early developmental stages.

Finally, this invention is also based on sequence alignments between plant species to identify the subfamily of GDSL-motif lipase-acylhydrolase proteins involved in fruit development, particularly in fruit cuticle polymerization.

Plants of the invention with reduced cuticle around the fruits have the advantage that they allow more cost-effective production of juice, puree and coulis of fruits.

Plants of the invention with more brilliant cuticle around the fruits have also the advantage to be more attractive for consumers.

Thus, a first object of the invention is an isolated plant GDSL-lipase polypeptide differentially expressed between the different steps of the plant development, preferably of the fruit development, and/or the different tissues of the plant, preferably of the fruit, comprising the sequences SEQ ID NO:1, SEQ NO:2, SEQ ID NO:3, SEQ ID NO:4 and SEQ ID NO:5 in this order from the N-terminal to the C-terminal ends

A second object of the invention is an isolated polynucleotide comprising a nucleic acid molecule encoding for a polypeptide of the invention, analogs, paralogs or orthologs thereof.

A third object of the invention is a vector comprising a polynucleotide of the invention.

A fourth object of the invention is a host cell genetically engineered with the polynucleotide of the invention, or with the vector of the invention.

A fifth object of the invention is a monoclonal antibody directed to a polypeptide of the invention.

A sixth object of the invention is a plant or part of plant genetically engineered to not express at least partially an isolated plant GDSL-lipase polypeptide of the invention.

A seventh object of the invention is a tomato plant or a part of a tomato plant genetically engineered to not express at least partially an isolated plant GDSL-lipase polypeptide selected from the group consisting of GDSL1 (SEQ ID NO:6), GDSL2 (SEQ ID NO:7) or GDSL3 (SEQ ID NO:8).

An eighth object of the invention is a tomato fruit genetically engineered to not express at least partially GDSL1 (SEQ ID NO:6).

A ninth object of the invention is a method for obtaining a plant mutant carrying at least a point mutation which suppresses at least partially the expression of an isolated plant GDSL-lipase polypeptide of the invention, said method comprising the following steps:
a. A mutagenesis treatment of seeds with a chemical mutagen such as Ethyl methanesulfonate (EMS);
b. Self-fertilization of the resulting M1 plants;
c. Extraction of DNA samples of the M2 individuals for mutational screening;
d. The DNA samples are pooled and arrayed in microtiter plates, and the pools are amplified using primers specific of a polypeptide of the invention;
e. Amplification products are incubated with the CEL I or ENDO I endonuclease;
f. Cleavage products are electrophoresed to identify punctual mutations in said polypeptide.

A tenth object of the invention is the use of a part of plant which is a fruit genetically engineered to not express at least partially an isolated plant GDSL-lipase polypeptide of the invention or a part of a plant mutant which is a fruit carrying at least a point mutation which suppresses at least partially the expression of an isolated plant GDSL-lipase polypeptide of the invention obtainable by the TILLING method of the invention for preparing puree, juice or coulis of said fruit.

An eleventh object of the invention is the use of a part of plant genetically engineered to not express at least partially an isolated plant GDSL-lipase polypeptide of the invention or a part of a plant mutant carrying at least a point mutation which suppresses at least partially the expression of an isolated plant GDSL-lipase polypeptide of the invention obtainable by the TILLING method of the invention for preparing an extract.

An twelfth object of the invention is the use of a tomato fruit genetically engineered to not express at least partially an isolated plant GDSL-lipase polypeptide of the invention or a tomato fruit carrying at least a point mutation which suppresses at least partially the expression of an isolated plant GDSL-lipase polypeptide of the invention obtainable by the TILLING method of the invention for preparing canned peeled tomatoes, tomato juice, tomato paste and tomato ketchup.

### DETAILED DESCRIPTION OF THE INVENTION

Polypeptides

The first object of the invention relates to an isolated plant GDSL-lipase polypeptide differentially expressed between the different steps of the plant development, preferably of the fruit development and/or the different tissues of the plant, preferably of the fruit, comprising the sequences SEQ ID NO:1, SEQ NO:2, SEQ ID NO:3, SEQ ID NO:4 and SEQ ID NO:5 in this order from the N-terminal to the C-terminal ends, and its orthologs, and derivatives thereof.

The terms “polypeptide”, “peptide” or “protein” are used interchangeable throughout this specification.

The isolated plant GDSL-lipase polypeptides according to the invention thus comprise five conserved blocks defined as SEQ ID NO:1 to 5.This is illustrated by the sequence alignments of FIGS. 10 and 11. The isolated plant GDSL-lipase polypeptides according to the invention have also a common function which is defined as a differential expression between the different steps of the plant development, preferably of the fruit development and/or the different tissues of the plant, preferably of the fruit.

Preferably, SEQ ID NO:1 is SEQ ID NO:9, more preferably is SEQ ID NO:29.

Preferably, SEQ ID NO:2 is SEQ ID NO:10, more preferably is SEQ ID NO:14.

Preferably, SEQ ID NO:3 is SEQ ID NO:11, more preferably is SEQ ID NO:15, even more preferably SEQ ID NO:17. In one embodiment, SEQ ID NO:3 is SEQ ID NO:30.

Preferably, SEQ ID NO:4 is SEQ ID NO:12, more preferably is SEQ ID NO:16.

Preferably, SEQ ID NO:5 is SEQ ID NO:13.

Preferably, the isolated plant GDSL-lipase polypeptide of the invention comprises the sequences SEQ ID NO:9, SEQ NO:10, SEQ ID NO:11, SEQ ID NO:12 and SEQ ID NO:13 in this order from the N-terminal to the C-terminal ends.

Preferably, the isolated plant GDSL-lipase polypeptide of the invention comprises the sequences SEQ ID NO:32, SEQ NO:14, SEQ ID NO:33, SEQ ID NO:16 and SEQ ID NO:13 in this order from the N-terminal to the C-terminal ends.

Preferably, the isolated plant GDSL-lipase polypeptide of the invention comprises a Y amino acid in the sequence separating SEQ ID NO:1 and SEQ ID NO:2, more preferably a PYG triad (SEQ ID NO:18), even more preferably SEQ ID NO:23.

Preferably, the isolated plant GDSL-lipase polypeptide of the invention comprises SEQ ID NO:19 in the sequence separating SEQ ID NO:2 and SEQ ID NO:3, more preferably SEQ ID NO:20 or SEQ ID NO:21.

Preferably, the isolated plant GDSL-lipase polypeptide of the invention comprises a CC duplicate in the sequence separating SEQ ID NO:4 and SEQ ID NO:5, more preferably SEQ ID NO:22.

Preferably, the isolated plant GDSL-lipase polypeptide of the invention comprises the sequences SEQ ID NO:1, SEQ ID NO:18, SEQ NO:2, SEQ ID NO:19, SEQ ID NO:3, SEQ ID NO:4, SEQ ID NO:22 and SEQ ID NO:5 in this order from the N-terminal to the C-terminal ends.

More preferably, the isolated plant GDSL-lipase polypeptide of the invention comprises the sequences SEQ ID NO:9, SEQ ID NO:23, SEQ NO:10, SEQ ID NO:20 or 21, SEQ ID NO:11, SEQ ID NO:12, SEQ ID NO:22 and SEQ ID NO:13 in this order from the N-terminal to the C-terminal ends.

In one embodiment, an isolated plant GDSL-lipase polypeptide according to the invention comprises the sequences SEQ ID NO:1, SEQ NO:2, SEQ ID NO:3, SEQ ID NO:4 and SEQ ID NO:5 in this order from the N-terminal to the C-terminal ends separated by sequences which present at least 60%, preferably at least 70%, and more preferably at least 80%, the most preferably at least 90% with the sequence SEQ ID NO:6, SEQ ID NO:7 or SEQ ID NO:8 its orthologs, and derivatives thereof. To determine the percent homology of two amino acid sequences (e.g., one of the sequences encoded by a nucleic acid of the invention and a mutant form thereof) or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=numbers of identical positions/total numbers of positions×100).

Preferably, an isolated plant GDSL-lipase polypeptide according to the invention comprises:

(a) a polypeptide sequence selected in the group consisting of SEQ ID NO:6, SEQ ID NO:7 or SEQ ID NO:8, or (b) a polypeptide sequence which is at least 60%, preferably at least 70%, and more preferably at least 80%, the most preferably at least 90% identical to the polypeptide of (a), said polypeptide being differentially expressed between the different steps of the plant development, preferably of the fruit development and/or the different tissues of the plant, preferably of the fruit, or

(c) a polypeptide sequence being a fragment of any one of (a) to (b), wherein said fragment or biologically active portion thereof is differentially expressed between the different steps of the plant development, preferably of the fruit development and/or the different tissues of the plant, preferably of the fruit.

More preferably, an isolated plant GDSL-lipase polypeptide according to the invention comprises:

(a) the polypeptide sequence SEQ ID NO:6, or

(b) a polypeptide sequence which is at least 60%, preferably at least 70%, and more preferably at least 80%, the most preferably at least 90% identical to the polypeptide of (a), said polypeptide being capable of altering cutin polymerization, or

(c) a polypeptide sequence which is at least 60%, preferably at least 70%, and more preferably at least 80%, the most preferably at least 90% identical to the polypeptide of (a), said polypeptide having an esterase activity, or

(d) a polypeptide sequence being a fragment of any one of (a) to (c), wherein said fragment or biologically active portion thereof is capable of altering cutin polymerization, or

(e) a polypeptide sequence being a fragment of any one of (a) to (c), wherein said fragment or biologically active portion thereof has an esterase activity.

A polypeptide according to the invention may be produced by culturing a cell transformed with a nucleotide sequence of this invention (in the sense orientation), allowing the cell to synthesize the protein and then isolating the protein, either as a free protein or as a fusion protein, depending on the cloning protocol used, from either the culture medium or from cell extracts. Alternatively, the protein can be produced in a cell-free system.

Preferably, an isolated plant GDSL-lipase polypeptide is GDSL1 of SEQ ID NO:6

Preferably, an isolated plant GDSL-lipase polypeptide is GDSL2 of SEQ ID NO:7

Preferably, an isolated plant GDSL-lipase polypeptide is GDSL3 of SEQ ID NO:8

Preferably, a polypeptide according to the invention has a lipase, hydrolase and/or esterase activity.

More preferably, an isolated plant GDSL-lipase polypeptide according to the invention has an esterase activity.

Table B Summarizes SEQ ID NO:1 to 22 with X is Any One of Amino Acids Except when X is Specified Below

| | | |
|---|---|---|
| BLOC 1 | SEQ ID NO: 1 | XGDSLXDXG |
| | (the first X is F or L) | F |
| | | L |
| | SEQ ID NO: 9 | FGDSLVDSG |
| | SEQ ID NO: 29 | RAFFVFGDSLVDSGNN |
| | SEQ ID NO: 18 | PYG |
| | SEQ ID NO: 23 | RADXPPYG |
| BLOC 2 | SEQ ID NO: 2 | TGRXXXGXXXXD |
| | | FSN |
| | | CCD |
| | | Y |
| | SEQ ID NO: 10 | TGRXSNGXXXXD |
| | | F I |
| | | C H |
| | SEQ ID NO: 14 | TGRFSNGXNIXD |
| | SEQ ID NO: 19 | GXNXAXAXXXXL |
| | | F S G |
| | | Y V A |
| | SEQ ID NO: 20 | GXNFASAGIGIL |
| | SEQ ID NO: 21 | GXNFAVAGATAL |
| BLOC 3 | SEQ ID NO: 3 | XXXX |
| | | GGND |
| | | SDE |
| | SEQ ID NO: 11 | GGNDXXN |
| | SEQ ID NO: 30 | GGNDFVNN |
| | SEQ ID NO: 15 | LVLXXLGGNDXXN |
| | SEQ ID NO: 17 | LVNXALVLXTXGGNDFVNNY |
| BLOC 4 | SEQ ID NO: 4 | XGAXRXXVXXXXPXGCXP |
| | | V |
| | | I |
| | SEQ ID NO: 12 | LGAXRVXVXXXXPLGCXP |
| | SEQ ID NO: 16 | LGARXVXVTGTGPLGC |
| | SEQ ID NO: 22 | ACCGXG |
| BLOC 5 | SEQ ID NO: 5 | CXXXXXXXXWDXXHXX |
| | | N PS |
| | | D LT |
| | SEQ ID NO: 13 | LCPNRXXYAFWDXFHPXEXA |

Table A Shows the Positions of SEQ ID NO:1 to 5 in GDSL1, GDSL2 and GDSL3.

| | Position of SEQ ID NO: | | | | |
|---|---|---|---|---|---|
| Protein | 1 | 2 | 3 | 4 | 5 |
| GDSL1 | 29-37 | 63-74 | 163-166 | 206-223 | 313-328 |
| GDSL2 | 38-46 | 74-85 | 173-176 | 211-228 | 331-346 |
| GDSL3 | 52-60 | 89-100 | 188-191 | 226-243 | 342-357 |

Expression

In the context of the present application, “differentially expressed” means an expression different in terms of cDNA level, mRNA level or protein level.

The different steps of the plant development comprise germination, stem elongation, flowering, fruit and seed or grain maturation, ear emergence in cereals.

The different steps of the fruit development are expressed in “days post anthesis” (DPA). The early step fruit development is approximately between 1 and 12 DPA.

The maturation step fruit development is approximately between 13 and 25 DPA.

The end of the fruit development is approximately between 35 and 45 DPA.

The different tissues of the plant comprise vegetative tissues and reproductive organs, in particular fruit, inflorescence, anther, petal, sepal, young leaf, mature leaf, stem, hair and root.

The different fruit tissues comprise seed, locular tissue, columella, mesocarp and exocarp.

Preferably, an isolated plant GDSL-lipase polypeptide according to the invention is differentially expressed during the fruit development. More preferably, an isolated plant GDSL-lipase polypeptide according to the invention is expressed during early fruit development or during fruit ripening.

Preferably, an isolated plant GDSL-lipase polypeptide according to the invention is specifically expressed in one or two fruit tissue(s). More preferably, an isolated plant GDSL-lipase polypeptide according to the invention is specifically expressed in the exocarp of the fruit or in the grains.

Preferably, an isolated plant GDSL-lipase polypeptide according to the invention is excreted outside the cell where it is expressed.

Species

Preferably, an isolated plant GDSL-lipase polypeptide according to the invention is isolated from a commercially important crop including Asteraceae (including the food crops lettuce, chicory, globe artichoke, sunflower, yacon, safflower), Cucurbitaceae (commonly known as gourds or cucurbits and includes crops like cucumbers, squashes (including pumpkins), luffas, melons and watermelons), Brassica (including swedes, turnips, kohlrabi, cabbages, brussels sprouts, cauliflower, broccoli, mustard seed and oilseed rape), Leguminous Crops (including dry beans, dry broad beans, dry peas, chickpea, garbanzo, bengal gram dry cowpea, black-eyed pea, blackeye bean, pigeon pea, toor, cajan pea, congo bean, lentil, bambara groundnut, earth pea vetch, common vetch Lupins, soybean, peanut) Chenopodiaceae (spinach). Liliaceae (unions, leek), Apiaceae (carrots), Grain crops (rice, barley, wheat, oats, corn), Solanaceae (including tomato, courgette, aubergine, hot pepper, sweet pepper, eggplant), fruit trees (apple tree, orange tree, Clementine tree), pine tree or vineyard.

More preferably, an isolated plant GDSL-lipase polypeptide according to the invention is isolated from a fruit-tree, from a cereal plant, for example cotton plant, rice plant, corn plant, oats plant, wheat plant, from vegetable plants, for example lettuce plant, courgette plant, aubergine plant, tomato plant, hot pepper plant or sweet pepper plant and from pine tree, vineyard.

Polynucleotides

Another object of the invention is an isolated polynucleotide comprising a nucleic acid molecule encoding for a polypeptide of the invention.

Preferably, an insolated polynucleotide of the invention comprises SEQ ID NO:24 encoding SEQ ID NO:6, analogs, paralogs or orthologs thereof.

Preferably, an insolated polynucleotide of the invention comprises SEQ ID NO:25 encoding SEQ ID NO:7, analogs, paralogs or orthologs thereof.

Preferably, an insolated polynucleotide of the invention comprises SEQ ID NO:26 encoding SEQ ID NO:8, analogs, paralogs or orthologs thereof.

As used herein, the term “nucleic acid molecule” is intended to include DNA molecules (e.g., cDNA or genomic DNA) and RNA molecules (e.g., mRNA) and analogs of the DNA or RNA generated using nucleotide analogs. This term also encompasses untranslated sequence located at both the 3′ and 5′ ends of the coding region of a gene: at least about 1000 nucleotides of sequence upstream from the 5′ end of the coding region and at least about 200 nucleotides of sequence downstream from the 3′ end of the coding region of the gene. The nucleic acid molecule can be single-stranded or double-stranded, but preferably is double-stranded DNA. An “isolated” nucleic acid molecule is one, which is substantially separated from other nucleic acid molecules, which are present in the natural source of the nucleic acid. Preferably, an “isolated” nucleic acid is substantially free of sequences that naturally flank the nucleic acid (i.e., sequences located at the 5′ and 3′ ends of the nucleic acid) in the genomic DNA of the organism, from which the nucleic acid is derived.

As used herein, the term “orthologs” refers to two nucleic acids from different species, but that have evolved from a common ancestral gene by speciation. Normally, orthologs encode proteins having the same or similar functions.

The term “polynucleotide” as used in accordance with the present invention relates to a polynucleotide comprising a nucleic acid molecule which encodes a polypeptide having a biologically activity as specified above.

Preferably, the polynucleotide of the invention comprises a sequence selected from the group consisting of SEQ ID NO:24, SEQ ID NO:25 or SEQ ID NO:26. Moreover, the term “polynucleotide” as used in accordance with the present invention further encompasses variants of the aforementioned specific polynucleotides. Said variants may represent orthologs, paralogs or other homologs of the polynucleotide of the present invention. The polynucleotide variants, preferably, also comprise a nucleic acid sequence characterized in that the sequence can be derived from the aforementioned specific nucleic acid sequences shown in SEQ ID NO:24, SEQ ID NO:25 or SEQ ID NO:26 by at least one nucleotide substitution, addition and/or deletion whereby the variant nucleic acid sequence shall still encode a polypeptide having a biological activity as specified above. Variants also encompass polynucleotides comprising a nucleic acid sequence which is capable of hybridizing to the aforementioned specific nucleic acid sequences, preferably, under stringent hybridization conditions. These stringent conditions are known to the skilled worker and can be found in Current Protocols in Molecular Biology, John Wiley & Sons, N. Y. (1989), 6.3.1-6.3.6. A preferred example for stringent hybridization conditions are hybridization conditions in 6× sodium chloride/sodium citrate (=SSC) at approximately 45° C., followed by one or more wash steps in 0.2×SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1 to 5×SSC (pH 7.2). If organic solvent is present in the abovementioned buffer, for example 50% formamide, the temperature under standard conditions is approximately 42° C. The hybridization conditions for DNA:DNA hybrids are, preferably, 0.1×SSC and 20° C. to 45° C., preferably between 30° C. and 45° C. The hybridization conditions for DNA: RNA hybrids are, preferably, 0.1×SSC and 30<0>C to 55° C., preferably between 45° C. and 55° C. The abovementioned hybridization temperatures are determined for example for a nucleic acid with approximately 100 by (=base pairs) in length and a G+C content of 50% in the absence of formamide. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Alternatively, polynucleotide variants are obtainable by PCR-based techniques such as mixed oligonucleotide primer-based amplification of DNA, i.e. using degenerated primers against conserved domains of the polypeptides of the present invention.

Further, variants include polynucleotides comprising nucleic acid sequences which are at least 70%, at least 75%, at least 80%, at least 85%, at least 90%, at least 95%, at least 98% or at least 99% identical to the nucleic acid sequences shown in SEQ ID NO:24, SEQ ID NO:25 or SEQ ID NO:26. A polynucleotide comprising a fragment of any of the aforementioned nucleic acid sequences is also encompassed as a polynucleotide of the present invention. The fragment shall encode a polypeptide which still has a biological activity as specified above. A fragment as meant herein, preferably, comprises at least 20, at least 50, at least 100, at least 250 or at least 500 consecutive nucleotides of any one of the aforementioned nucleic acid sequences or encodes an amino acid sequence comprising at least 20, at least 30, at least 50, at least 80, at least 100 or at least 150 consecutive amino acids of any one of the aforementioned amino acid sequences. Variant polynucleotides as referred to in accordance with the present invention may be obtained by various natural as well as artificial sources. For example, polynucleotides may be obtained by in vitro and in vivo mutagenesis approaches using the above mentioned mentioned specific polynucleotides as a basis. Moreover, polynucleotide being homologs or orthologs may be obtained from various plant species.

The present invention, thus, also encompasses an oligonucleotide which specifically binds to the polynucleotides of the present invention. Binding as meant in this context refers to hybridization by Watson-Crick base pairing discussed elsewhere in the specification in detail. An oligonucleotide as used herein has a length of at most 100, at most 50, at most 40, at most 30 or at most 20 nucleotides in length which are complementary to the nucleic acid sequence of the polynucleotides of the present invention. The sequence of the oligonucleotide is, preferably, selected so that a perfect match by Watson-Crick base pairing will be obtained. The oligonucleotides of the present invention may be suitable as primers for PCR-based amplification techniques. Moreover, the oligonucleotides may be used for RNA interference (RNAi) approaches in order to modulate and, preferably down- regulate, the activity of the polypeptides encoded by the polynucleotides of the present invention. As used herein, the term “RNA interference (RNAi)” refers to selective intracellular degradation of RNA used to silence expression of a selected target gene, i.e. the polynucleotide of the present invention. RNAi is a process of sequence-specific, post- transcriptional gene silencing in organisms initiated by double-stranded RNA (dsRNA) that is homologous in sequence to the gene to be silenced. The RNAi technique involves small interfering RNAs (siRNAs) that are complementary to target RNAs (encoding a gene of interest) and specifically destroy the known mRNA, thereby diminishing or abolishing gene expression. RNAi is generally used to silence expression of a gene of interest by targeting mRNA, however, any type of RNA is encompassed by the RNAi methods of the invention. Briefly, the process of RNAi in the cell is initiated by long double stranded RNAs (dsRNAs) being cleaved by a ribonuclease, thus producing siRNA duplexes. The siRNA binds to another intracellular enzyme complex which is thereby activated to target whatever mRNA molecules are homologous (or complementary) to the siRNA sequence. The function of the complex is to target the homologous mRNA molecule through base pairing interactions between one of the siRNA strands and the target mRNA. The mRNA is then cleaved approximately 12 nucleotides from the 3′ terminus of the siRNA and degraded. In this manner, specific mRNAs can be targeted and degraded, thereby resulting in a loss of protein expression from the targeted mRNA. A complementary nucleotide sequence as used herein refers to the region on the RNA strand that is complementary to an RNA transcript of a portion of the target gene. The term “dsRNA” refers to RNA having a duplex structure comprising two complementary and anti-parallel nucleic acid strands. Not all nucleotides of a dsRNA necessarily exhibit complete Watson-Crick base pairs; the two RNA strands may be substantially complementary. The RNA strands forming the dsRNA may have the same or a different number of nucleotides, with the maximum number of base pairs being the number of nucleotides in the shortest strand of the dsRNA. Preferably, the dsRNA is no more than 49, more preferably less than 25, and most preferably between 19 and 23, nucleotides in length. dsRNAs of this length are particularly efficient in inhibiting the expression of the target gene using RNAi techniques. dsRNAs are subsequently degraded by a ribonuclease enzyme into short interfering RNAs (siRNAs). RNAi is mediated by small interfering RNAs (siRNAs). The term “small interfering RNA” or “siRNA” refers to a nucleic acid molecule which is a double stranded RNA agent that is complementary to i.e., able to base-pair with, a portion of a target RNA (generally mRNA), i.e. the polynucleotide of the present invention being RNA. siRNA acts to specifically guide enzymes in the host cell to cleave the target RNA. By virtue of the specificity of the siRNA sequence and its homology to the RNA target, siRNA is able to cause cleavage of the target RNA strand, thereby inactivating the target RNA molecule. Preferably, the siRNA which is sufficient to mediate RNAi comprises a nucleic acid sequence comprising an inverted repeat fragment of the target gene and the coding region of the gene of interest (or portion thereof). Also preferably, a nucleic acid sequence encoding a siRNA comprising a sequence sufficiently complementary to a target gene is operatively linked to a expression control sequence. Thus, the mediation of RNAi to inhibit expression of the target gene can be modulated by said expression control sequence. Preferred expression control sequences are those which can be regulated by a exogenous stimulus, such as the tet operator whose activity can be regulated by tetracycline or heat inducible promoters. Alternatively, an expression control sequence may be used which allows tissue- specific or expression of the siRNA or expression at defined timepoints in development. The complementary regions of the siRNA allow sufficient hybridization of the siRNA to the target RNA and thus mediate RNAi. In mammalian cells, siRNAs are approximately 21-25 nucleotides in length (see Tuschl et al. 1999 and Elbashir et al. 2001). The siRNA sequence needs to be of sufficient length to bring the siRNA and target RNA together through complementary base-pairing interactions. The siRNA used with a seed specific expression system e.g. under control of the USP promoter of the invention may be of varying lengths. The length of the siRNA is preferably greater than or equal to ten nucleotides and of sufficient length to stably interact with the target RNA; specifically 15-30 nucleotides; more specifically any integer between 15 and 30 nucleotides, most preferably 15, 16, 17, 18, 19, 20, 21, 22, 23, 24, 25, 26, 27, 28, 29, and 30. By “sufficient length” is meant an oligonucleotide of greater than or equal to 15 nucleotides that is of a length great enough to provide the intended function under the expected condition. By “stably interact” is meant interaction of the small interfering RNA with target nucleic acid (e.g., by forming hydrogen bonds with complementary nucleotides in the target under physiological conditions). Generally, such complementarity is 100% between the siRNA and the RNA target, but can be less if desired, preferably 91%, 92%, 93%, 94%, 95%, 96%, 97%, 98%, or 99%. For example, 19 bases out of 21 bases may be base-paired. In some instances, where selection between various allelic variants is desired, 100% complementary to the target gene is required in order to effectively discern the target sequence from the other allelic sequence. When selecting between allelic targets, choice of length is also an important factor because it is the other factor involved in the percent complementary and the ability to differentiate between allelic differences. Preferably, the oligonucleotides used for RNAi approaches are selected from the 5<′>or 3′ untranslated region of a cDNA corresponding to a polynucleotide of the present invention.

Also in a preferred embodiment, the oligonucleotide of the present invention can be used for the generation of or as a micro RNA (miRNA). These miRNAs are single-stranded RNA molecules of preferably 20 to 25, more preferably 21 to 23 nucleotides in length capable of regulating gene expression. miRNAs are physiologically encoded by genes that are transcribed from DNA but not translated into protein (non-coding RNA). Rather, they are processed from primary transcripts known as pri-miRNA to short stem-loop structures called pre-miRNA and finally to functional miRNA. The mature miRNA molecules are partially complementary to one or more messenger RNA (mRNA) molecules. Due to this complementary regions, they are capable of binding to a given target mRNA and to subsequently down-regulate expression thereof. Preferably, miRNAs to be used in accordance with the present invention comprise sequence stretches complementary to the polynucleotide sequences of the present invention referred to above. More preferably, the sequence stretches are between 20 and 30 nucleotides, more preferably, between 20 and 25 nucleotides and, most preferably, between 21 and 23 nucleotides in length. The miRNAs, preferably, are capable of binding to complementary sequences of the coding regions or of the 3<′> or the 5<′>UTR sequences of the polynucleotides of the invention.

More preferably, the oligonucleotide of the present invention, thus, comprises a sequence of at least 15 nucleotides in length complementary to the nucleic acid sequence of the polynucleotide of the invention. Most preferably, the said oligonucleotide is capable of down regulating the expression of the said polynucleotide, preferably either by functioning as a double stranded RNAi molecule or as a single stranded miRNA molecule. Downregulation as meant herein relates to a statistically significant reduction of the mRNA detectable in a cell, tissue or organism or even to a failure to produce mRNA in detectable amounts at all. This also includes the reduction of of the stability of mRNA encoding all or a part of any sequence described herein. Moreover, downregulation also encompasses an impaired, i.e. significantly reduced, production of protein from RNA sequences encoding all or a part of any sequence or even the absence of detectable protein production.

The present invention provides antisense oligonucleotides and polynucleotides complementary to an isolated plant GDSL-lipase polypeptide according to the invention or one of its orthologs from another plant which hybridizes with it under high stringency conditions. Such antisense oligonucleotides should be at least about six nucleotides in length to provide minimal specificity of hybridization and may be complementary to one strand of DNA or mRNA encoding an isolated plant GDSL-lipase polypeptide according to the invention or a portion thereof, or to flanking sequences in genomic DNA which are involved in regulating the expression of said isolated plant GDSL-lipase polypeptide according to the invention. The antisense oligonucleotide may be as large as 100 nucleotides and may extend in length up to and beyond the full coding sequence for which it is antisense. The antisense oligonucleotides can be DNA or RNA or chimeric mixtures or derivatives or modified versions thereof, single stranded or double stranded. The action of the antisense oligonucleotide may result in alteration, primarily inhibition, of the expression of said isolated plant GDSL-lipase polypeptide according to the invention in cells.

SiRNAs used by the inventors to inhibit the expression of GDSL1 (SEQ ID NO:6) have the sequences SEQ ID NO:49 and SED ID NO:50 and are also an object of the present invention.

Vector

A vector according to the invention comprises a polynucleotide according to the invention.

Vectors can be plasmids preferably, or may be viral or other vectors known in the art to replicate and express genes encoded thereon in plant cells or bacterial cells. The vector becomes chromosomally integrated such that it can be transcribed to produce the desired antisense senescence-induced lipase RNA. Such plasmid or viral vectors can be constructed by recombinant DNA technology methods that are standard in the art. For example, the vector may be a plasmid vector containing a replication system functional in a prokaryotic host. Alternatively, the vector may be a plasmid containing a replication system functional in Agrobacterium.

In order for a newly inserted gene or DNA sequence to be expressed, resulting in production of the protein which it encodes, or in the case of antisense DNA, to be transcribed, resulting in an antisense RNA molecule, the proper regulatory elements should be present in proper location and orientation with respect to the gene or DNA sequence. The regulatory regions may include a promoter, a 5′-non-translated reader sequence and a 3′-polyadenylation sequence as well as enhancers and other regulatory elements.

Promoter regulatory elements that are useful in the vector according to the invention in combination with a nucleic acid according to the invention include any plant promoter in general.

The vector of the present invention, preferably, further comprises selectable markers for propagation and/or selection in a host. The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion. Vectors can be introduced into prokaryotic or eukaryotic cells via conventional transformation or transfection techniques.

Host Cells

A host cell according to the invention is genetically engineered with a polynucleotide of the invention or with a vector of the invention.

Host cells are primary cells or cell lines derived from multicellular organisms such as plants or animals. Furthermore, host cells encompass prokaryotic or eukaryotic single cell organisms (also referred to as microorganisms), e.g. bacteria or fungi including yeast or bacteria. Primary cells or cell lines to be used as host cells in accordance with the present invention may be derived from the multicellular organisms, preferably from plants.

Also provided in accordance with the present invention is a method for the manufacture of a polypeptide having the biological activity of a polypeptide encoded by a polynucleotide of the present invention comprising:

(a) expressing the polynucleotide of the present invention in a host cell; and

(b) obtaining the polypeptide encoded by said polynucleotide from the host cell.

The polypeptide may be obtained, for example, by all conventional purification techniques including affinity chromatography, size exclusion chromatography, high pressure liquid chromatography (HPLC) and precipitation techniques including antibody precipitation. It is to be understood that the method may - although preferred -not necessarily yield an essentially pure preparation of the polypeptide. It is to be understood that depending on the host cell which is used for the aforementioned method, the polypeptides produced thereby may become post-translationally modified or processed otherwise.

Antibodies

The invention also relates to a monoclonal antibody directed to a polypeptide according to the invention.

Antibodies against the polypeptides of the invention can be prepared by well known methods using a purified polypeptide according to the invention or a suitable fragment derived therefrom as an antigen. A fragment which is suitable as an antigen may be identified by antigenicity determining algorithms well known in the art. Such fragments may be obtained either from the polypeptide of the invention by proteolytic digestion or may be a synthetic peptide. Preferably, the antibody of the present invention is a monoclonal antibody, a polyclonal antibody, a single chain antibody, a human or humanized antibody or primatized, chimerized or fragment thereof. Also comprised as an antibody by the present invention is a bispecific antibody, a synthetic antibody, an antibody fragment, such as Fab, Fv or scFv fragments etc., or a chemically modified derivative of any of these. The antibody of the present invention shall specifically bind to the polypeptide of the invention, i.e. it shall not significantly cross react with other polypeptides or peptides. Specific binding can be tested by various well known techniques. Antibodies or fragments thereof can be obtained by using methods which comprise the fusion of mouse myeloma cells to spleen cells derived from immunized mammals. The antibodies can be used, for example, for the immunoprecipitation, immunolocalization or purification (e.g., by affinity chromatography) of the polypeptides of the invention as well as for the monitoring of the presence of said variant polypeptides, for example, in recombinant organisms, and for the identification of compounds interacting with the proteins according to the invention.

Plant

The invention also relates to a plant or part of plant which does not express at least partially an isolated plant GDSL-lipase polypeptide according to the invention. Preferably, the plant or part of plant according to the invention is genetically engineered to not express at least partially an isolated plant GDSL-lipase polypeptide according to the invention.

The quantity of expressed GDSL-lipase polypeptide according to the invention can be decreased by a decrease in cDNA or RNA levels corresponding to the GDSL-lipase polypeptide according to the invention or by a decrease in protein level whereas the cDNA and RNA levels did not change.

In one embodiment, the GDSL-lipase polypeptide according to the invention level is decreased by RNAi.

The quantity of expressed GDSL-lipase polypeptide according to the invention can also be decreased by a decrease in cDNA or RNA or protein level of a precursor of said GDSL-lipase polypeptide according to the invention.

In one embodiment, the level of a precursor of a GDSL-lipase polypeptide according to the invention level is decreased by RNAi.

The invention also relates to a plant or part of plant which expresses an isolated plant GDSL-lipase polypeptide according to the invention having an abnormal activity.

By “a modified isolated plant GDSL-lipase polypeptide according to the invention to not have a normal activity” is meant:
an isolated plant GDSL-lipase polypeptide according to the invention the differential expression of which is altered, and/or
an isolated plant GDSL-lipase polypeptide according to the invention the role in cutin polymerization of which is altered, and/or
an isolated plant GDSL-lipase polypeptide according to the invention the esterase, lipase or hydrolase activity of which is altered.

In one embodiment, the polypeptide is genetically modified to have an abnormal activity. For example, a punctual mutation or a deletion or an insertion of nucleotides has been introduced in the part of the corresponding gene responsible of the activity and this modification implies an abnormal activity.

In another embodiment, another polypeptide involved in the activity pathway of the polypeptide of the invention is modified and this modification inhibits the activity of the polypeptide of the invention.

As used herein, the term “plant” refers to either a whole plant, a plant part, a plant cell or a group of plant cells.

Preferably, a plant according to the invention is a transgenic plant.

A transgenic plant is defined herein as a plant which is genetically modified in some way. The altered genetic material may encode a protein comprising a regulatory or control sequence or may be or include an antisense sequence or encode an antisense RNA or siRNA which inhibits the DNA or mRNA sequence or portion thereof responsible of the expression of the polypeptide of which a modification of the expression is foreseen.

A “transgene” or “transgenic sequence” is defined as a foreign gene or partial sequence which has been incorporated into a transgenic plant.

Transgenic plants made in accordance with the present invention may be prepared by DNA transformation using any method of plant transformation known in the art. Plant transformation methods include direct cocultivation of plants, tissues or cells with Agrobacterium tumefaciens or direct infection; direct gene transfer into protoplasts or protoplast uptake; electroporation; particle bombardment; injection into meristematic tissues of seedlings and plants; injection into protoplasts of cultured cells and tissues. Generally a complete plant is obtained from the transformation process. Plants are regenerated from protoplasts, callus, tissue parts or explants, etc.

Expression of an isolated plant GDSL-lipase polypeptide according to the invention can be assayed in a conventional immunoassay method using a specific antibody as described herein.

Preferably, a plant according to the invention is a tomato plant or a part of a tomato plant genetically engineered to not express at least partially an isolated plant GDSL-lipase polypeptide selected from the group consisting of GDSL1 (SEQ ID NO:6), GDSL2 (SEQ ID NO:7) or GDSL3 (SEQ ID NO:8).

More preferably, a plant according to the invention is a tomato fruit genetically engineered to not express at least partially GDSL1 (SEQ ID NO:6).

Another object of the present invention relates to a method for producing a transgenic plant, preferably a tomato plant, with reduced cuticle around the fruits. Transgenic plants may be obtained in various manners including e.g. silencing of the polynucleotide according to the invention by introduction of antisense, sense suppression or RNAi (RNA interference) nucleic acid constructs into the plants, or by knock-out of the polynucleotide according to the invention by homologous recombination. In another preferred embodiment, the transgenic plants with non-dehiscent anthers may be obtained by knock-out of the polynucleotide according to the invention by homologous recombination. Homologous recombination allows introduction in a genome of a selected nucleic acid at a defined selected position. Methods for performing homologous recombination in plants have been described not only for model plants (Offringa et al, 1990 EMBO J. 9(10):3077-84) but also for crop plants, for example rice (Iida and Terada, Curr Opin Biotechnol. 2004 April; 15(2): 132- 8).

TILLING

Another object of the present invention is a TILLING (Targeted Induced Local Lesions IN Genomes) method for obtaining a plant mutant carrying at least a point mutation which suppresses at least partially the expression of an isolated plant GDSL-lipase polypeptide according to the invention, said method comprising the following steps:
a. A mutagenesis treatment of seeds with a chemical mutagen such as Ethyl methanesulfonate (EMS);
b. Self-fertilization of the resulting M1 plants;
c. Extraction of DNA samples of the M2 individuals for mutational screening;
d. The DNA samples are pooled and arrayed in microtiter plates, and the pools are amplified using primers specific of a polypeptide as claimed in claims 1 to 6;
e. Amplification products are incubated with the CEL I or ENDO1 endonuclease;
f. Cleavage products are electrophoresed to identify punctual mutations in said polypeptide.

Methods for TILLING are well known in the art (McCallum et al reviewed by Stemple). The TILLING mutagenesis technology is useful to generate and/or identify, and to eventually isolate mutagenised variants of a polynucleotide according to the invention. TILLING also allows selection of plants carrying such mutant variants. TILLING combines high-density mutagenesis with high-throughput screening methods. The steps typically followed in TILLING are: (a) a mutagenesis treatment of seeds with a chemical mutagen such as Ethyl methanesulfonate (EMS) or N-ethyl-N-nitrosourea (ENU); (b) backcrossing the plants for at least one generation; (c) preparation of DNA from tissue samples of the plants and (d) pooling of DNAs from individuals; (e) PCR amplification of a region of interest, (f) detection of mutant PCR products (g) identification of the mutant individual and (h) optionally, sequencing of the mutant PCR. Initially in the method detection of mutant PCR products in step (f) and optionally (g) was performed by denaturation and annealing to allow formation of heteroduplexes and subsequent detection of heteroduplexes by DHPLC (Denaturing High Perfomance Liquid Chromatography). The method was made higher throughput by using the restriction enzyme CEL-I or ENDO-I combined with a gel based system to identify mutations (Colbert et al., 2001, Plant Physiol. 126(2):480-4). Other methods for detection or identification of single base mutations in the target gene that may be used in methods of TILLING include e.g. resequencing DNA as has been described (see e.g. Slade et al., 2005, Nat Biotechnol. 23(1):75-81) or massive parallel sequencing (e.g. of Roche/454 Life Sciences performed on a GS 20 Genome Sequencer) to produce sequence information for a given locus in thousands of individuals, optionally combined with a 3D pooling strategy (see e.g the Keypoint™ technology at www.keygene.com). Mutagenesis may be performed by radiation or with a chemical mutagen such as EMS (Lightner and Caspar, 1998, “Seed mutagenesis of Arabidopsis”, In: Methods in Molecular Biology: Arabidopsis Protocols (eds. J. M. Martinez-Zapater and J. Salinas), pp. 91-103. Totowa, N.J.: Humana Press) or ENU (Draper et al., 2004, Methods Cell Biol. 2004;77:91-1 12).

Preferably, the mutation in the GDSL1-allele cause the allele to be a truncation or missense allele.

Uses

Another object of the invention is the use of a part of plant which is a fruit genetically engineered to not express at least partially an isolated plant GDSL-lipase polypeptide of the invention or a part of a plant mutant which is a fruit carrying at least a point mutation which suppresses at least partially the expression of an isolated plant GDSL-lipase polypeptide of the invention obtainable by the TILLING method of the invention for preparing puree, juice or coulis of said fruit.

Preferably, said fruit is a tomato.

Another object of the invention is the use of a part of plant genetically engineered to not express at least partially an isolated plant GDSL-lipase polypeptide of the invention or a part of a plant mutant carrying at least a point mutation which suppresses at least partially the expression of an isolated plant GDSL-lipase polypeptide of the invention obtainable by the TILLING method of the invention for preparing an extract, preferably without using organic solvents.

Preferably, said use is the use of a part of plant according to the invention which is a wheat or barley grain for preparing malt.

Another object of the invention is the use of a tomato fruit genetically engineered to not express at least partially an isolated plant GDSL-lipase polypeptide of the invention or a tomato fruit carrying at least a point mutation which suppresses at least partially the expression of an isolated plant GDSL-lipase polypeptide of the invention obtainable by the TILLING method of the invention for preparing canned peeled tomatoes, tomato juice, tomato paste or tomato ketchup.

The different possible uses of the plants and part of plants of the invention are linked to their following advantages:
their visual aspect is modified so that they show a shiny appearance appealing to the consumer
their epiderm thickness is modified so that their sensorial quality as a fresh product is more attractive to the consumer—e.g. reducing the amount of peel in tomato fruit so that children are not rebutted by the feeling of thick fruit peel in their mouth when eating cherry tomato fruit
their epiderm thickness and composition are modified in order to facilitate the extraction of compounds of interest—e.g. reducing the cuticle thickness and composition on wheat or barley grains in order to facilitate the use of malt during the brewing process—e.g. reducing the cuticle thickness and composition so that the use of organic solvents is not necessary anymore
their epiderm thickness and composition are modified in order to reduce the amount of wastes (peel, cuticle)—e.g. reducing the amount of peel wastes generated when processing tomato to obtain canned peeled tomatoes, tomato juice, tomato paste and tomato ketchup
their epiderm thickness and composition are modified in order so that surface composition of the cuticle is modified and does not allow pathogens to attack the plant—e.g. suppression of host recognition by the pathogen in order to suppress host-pathogen interactions detrimental to the host plant

Having now generally described the invention, the same will be more readily understood through reference to the following figures and examples which are provided by way of illustration, and are not intended to be limiting to the present invention, unless specified.

### FIGURES

FIG. 1: Sequence alignment of the GDSL1, GDLS2 and GDSL3 proteins.

FIG. 2: Expression profiles of 3 genes coding for putative GDSL lipase in tomato plant organs. Real time RT-PCR experiments were performed to assess transcript abundance of GDSL1 (A), GDSL2 (B) and GDSL3 (C). IF, immature fruit; Sta, stamen; Pet, petals; YL, young leaves; ML, mature leaves; Ste, stem; Po, pollen; R, roots. AU=arbitrary units. Vertical bars represent standard deviation (n=3).

FIG. 3: Temporal expression of GDSL lipase coding genes along fruit development.

FIG. 4: Expression pattern of GDSL lipase coding genes in fruit tissues.

FIG. 5: Exocarp cuticule thickness in fruit of WT and P35SSlGDSL1 RNAi plants. (A) Cross section of exocarp on fruits from WT and transgenic lines 10, 4, 3 and 17 (light microscopy). Coloration with Sudan Red revealed cutine polymers and cutinised cells of the first exocarp cells layer encased in cutin. Arrows 1 and 2 represent the positions of thickness measurements. These measurements were performed (B) between two epidermic cells (widest part of the cuticle) or (C) above one epidermic cell (smallest part of the cuticle). Vertical bars represent standard deviation (n=60), * and *** indicate a significant difference of thickness with WT samples (t test, respectively P<0.05 and P<0.001).

FIGS. 6 and 7: ATR FTIR

FIG. 8: Hydrolytic activity of Thr-GDSL1 fusion protein (B.) and rGDSL1 recombinant protein (C.). Purified Thr-GDSL1 fusion protein and cleaved rGDSL1 protein were assayed for their hydrolytic activity on various para-nitrophenyl (pNP) derivates, pNP acetate (pNPA, C₂), pNP butyrate (pNPB, C₄), pNP laurate (pNPL, C₁₂), pNP myristolate (pNPM, C₁₄), and pNP palmitate (pNPP, C₁₆). Fungal cutinase was used as positive control and protein solubilisation buffer and protein extraction from pETD without insert were used as negative controls (A.). Measurements were performed after 120 min incubation at 37° C. by optical density reading at 405 nm with spectrophotometer.

FIG. 9: immunolocalization of GDSL in Wt tomato exocarp (A). Blank control without GDSL1 antibody

FIG. 10: Alignement GDSL1, 2 et 3 de Tomate avec 2 sequences de piment

FIG. 11: Alignement GDSL1, 2 et 3 de Tomate avec 3 sequences de Pomme de terre

### EXAMPLES

### Example: Isolation and Characterization of GDSL1

1. Material and Methods

1.1. Tomato Fruit Cuticle Isolation

Cuticles were isolated from commercial red ripe tomato fruits of Solanum lycopersicum. Isolation of cuticles was carried out using a modified protocol (Petracek and Bukovac, 1995). Briefly, peels were removed from tomato red fruits and incubated for two weeks at room temperature in an enzymatic solution of fungal cellulase (2% v/v, Sigma, St Louis, Mo., USA) and fungal pectinase (2% v/v, Sigma) containing 1 mM NaN₃ to prevent microbial growth. The enzymatic solution was changed four times during the 2 weeks. Cuticular matrices were finally separated from the epidermis cell walls by extensive washing in distilled water and dried on nylon tissue. Cuticles were scanned and their surface was assessed using the Image J software (http://rsb.info.nih.gov/ij/).

1.2. Cloning, Expression, Purification of Recombinant GDSL1

1.2.1. Cloning of GDSL1

The gds11 gene was overexpressed in Escherichia coli using pET expression system. Specific primers were designed (GDSL3′: 5′TAAAGCTTATGCATGTGAAT3′=SEQ ID NO:27 and GDSL5′: 5′ATCCATGGGGCAAAGTGAAGCTAGGGCATTT3′ =SEQ ID NO:28) and used to amplify and clone the coding region of gdsl1 with the NcoI and HindIII restriction sites (underlined sequences). cDNA was subcloned into expression vector pETD. The recombinant plasmid, pETD-GDSL1, was introduced into E. coli BL21 competent cells. E. coli transformed with pETD vector without insert was used as negative control in all study. Clones with plasmid were selected using 100 μg/ml ampicillin on Luria Bertani (LB) medium.

A second construction was also used to overexpress and purify GDSL1 in E Coli using the same pET expression system. Specific primer were designed (GDSL6 5′ ATCCATGGGGCAAAGTGAAGCTAGGGCATTT=SEQ ID n° 29) GDSL7 5′ATCTCGAGTGCATGTGAATCCATAGCCAG=SEQ ID n° 30) and used to amplify and clone the coding region of gdsl1 with the NcoI and XhoI restriction sites. Cloning and production of the protein was conducted in the same conditions as described above.

1.2.2. Production and Purification of Recombinant Protein in Escherichia Coli

For production, cultures were grown in LB medium added by 100 μg/ml ampicillin at 37° C. to early exponential phase (optical density at 600 nm [OD600] of 0.6), and then IPTG109 (isopropyl-β-D-thiogalactopyranoside) was added to a final concentration of 0.5 mM.

Subsequent protein expression was allowed to proceed for 3 h at 37° C. Bacterial cells were harvested by centrifugation at 14000 rpm for 20 min at 4° C. Dried pellets were stored at −20° C. until using.

Thioredoxin-GDSL1 (Thx-GDSL1) fusion protein was extracted from culture pellets in solubilized buffer (Na-Phosphate 20 mM, NaCl 500 mM and urea 8 M). After purification by metal chelate affinity chromatography with Ni—NTA His resin (Invitrogen), Thr-GDSL1 was submitted to chemical cleavage and recombinant GDSL1 (rGDSL1) protein then purified passing another time on nickel affinity chromatography.

For the second construction, the protein was extracted from culture pellets with a Tris NaCl (50 mM, 200 mM, pH7,5) buffer. Protein purification was conducted through a Ni—NTA resin.

1.3. Hydrolase Activities

1.3.1. Assay of Lipase Activity of GDSL1 Recombinant in E. Coli

To monitor esterase activity of GDSL1 in E. coli, a modified tributyrin agar diffusion assay (Rashid et al., 2001) was used. Briefly, LB agar medium containing tributyrin (1% v/v), IPTG (50 μM) and ampicillin (50 μg/mL) was emulsified by sonication after autoclaving and poured into Petri dishes. In this agar plates, several wells (5 mm diameter) were aseptically punched. Ten microliters of fresh culture were loaded in the wells and examined for their ability to form clear halos, indicating esterase activity. A culture of E. coli containing pETD without GDSL1 was used as negative control.

1.3.2. Lipase/Esterase Colorimetric Enzyme Assay

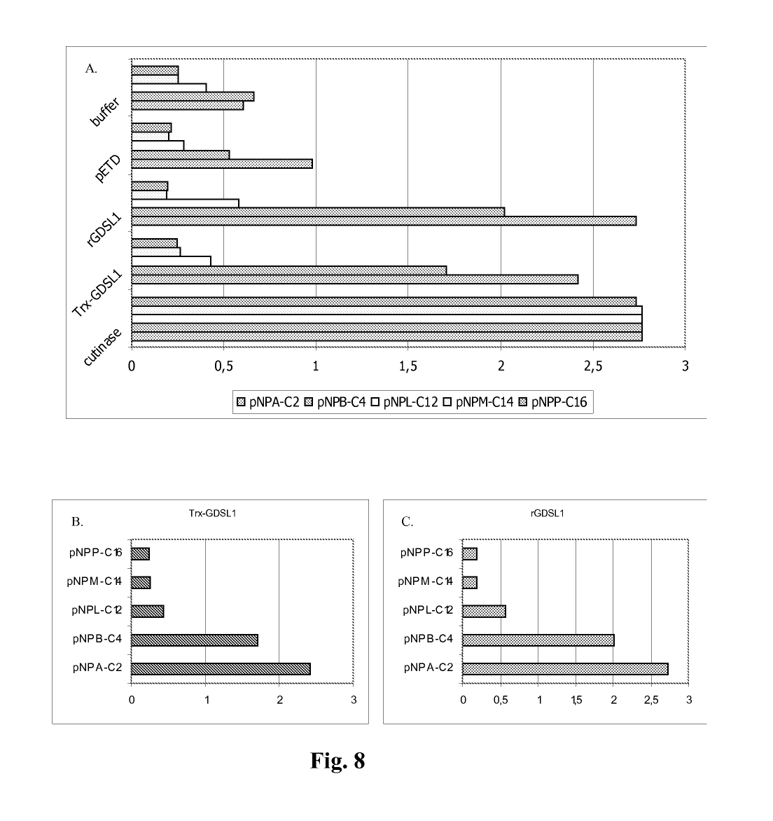

Lipase and esterase activity was assessed by measuring the absorbance of the pnitrophenol (pNP) (Ruiz et al., 2004). Different substrates were chosen as a function of their chain length: p-nitrophenyl acetate (pNPA, C2), p-nitrophenylbutyrate (pNPB, C4), pnitrophenyl laurate (pNPL, C12), p-nitrophenyl myristate (pNPM, C14) and p-nitrophenyl palmitate (pNPP, C16). Each substrate was dissolved at 20 mM in isopropanol and sonicated for 3 min. Each solution was diluted in phosphate-Triton X100 buffer in a ratio 1:10 (v/v) under gentle agitation to obtain a final concentration of 2 mM pNP, 10% isopropanol, 1,2% Triton X100 and 50 mM Na-phosphate buffer pH 7.8. 1 mL of this solution was mixed with 1 mL of enzyme solubilized in 20 mM Na-phophate pH 7.8 and 150 mM NaCl. 1 mL of pET32b in the same buffer and 1 mL of this buffer alone were used as negative control and 25 μL of cutinase from Fusarium graminearum and 975 μL of buffer as positive control. The absorbance of pNP was recorded at 405 nm for each samples during 30 min with measurement every 5 min. (see FIG. 8)

1.4. Plant Material and Growth Conditions

Cherry tomato plants, Solanum lycopersicum var. cerasiformae ‘West Virginia 106’, were grown under standard green house conditions. Medium-sized fruit tomato plants, Solanum lycopersicum var. Ailsa craig, were grown in a growth chamber with a light period of 14 h in light and 10 h in dark under a thermoperiod of 25° C./20° C. and watered with a nutrient solution three times a day. For in vitro culture, seeds were decontaminated in a calcium hypochlorite (4% p/v) and Tween 20 (0.1% v/v) solution for 20 min, rinsed in distilled water, dried at room temperature and sown in a Murashige and Skoog. (MS, 1.1 g/L) medium with agar (8 g/L) containing (transgenic plants) or not (wild type) kanamycin (300 μg/L).

1.5. Plant Transformation

cDNA fragment was obtained by reverse transcription (SuperScript II Reverse transcriptase, Invitrogen, life technology) on 2 μg of total RNA from tomato fruit. Two fragments of 509 by and 148 bp, located respectively in the 5′ region and in the 3′-untranslated region of GDSL1, were amplified by PCR using ExTaq DNA polymerase and gene specific primers (table I). Each purified DNA fragment was introduced in the Gateway system entry vector (pDONR 201) and then transferred as an inverted repeat under control of 35S promoter in the RNAi destination vector (pK7GWIWG2), which confers kanamycin resistance to transformed plants (Karimi et al., 2002). After verification by sequencing, constructions were used to transform cherry tomato plants ('West Virginia 106′) via Agrobacterium tumefaciens strain GV3101 (Hamza and Chupeau, 1993). Primary transfomant were used to analyze expression profile and homozygous plants were used to study phenotypic modifications.

TABLE I

| primer | 5′-3′ sequence |
|---|---|
| GDSL1s1 | AAAAAGCAGGCTCCAAAATGGCCACACCTACT |
| GDSL1s2 | AAAAAGCAGGCTTTTTTGTTGCTAATTTTTGCCTA |
| GDSL1as3 | AGAAAGCTGGGTTGACAAAAATCATTGCCTCCA |
| GDSL1as4 | AGAAAGCTGGGTCAACAATATCCACACTCCACCCTA |
| attB1 | GGGGACAAGTTTGTACAAAAAAGCAGGCTNN |
| attB2 | GGGGACCACTTTGTACAAGAAAGCTGGGTNN |

1.6. Gene Expression

1.6.1. Sequence Analysis

GDSL sequences are indexed in the Institute of Genomic Research (TIGR) Plant Transcript Assemblies Database (http://plantta.jcvi.org/search.shtml, Solanum lycopersicum, release 5, 01/06/2007) and in the SOL Genomics Network (http://www.sgn.cornell.edu/) under accession numbers as follows: GDSL1 (TA37529_{—}4081; SGN-U566206), GDSL2 (TA38323_{—}4081; SGN-U585129) and GDSL3 (TA41795_{—}4081; SGN-U318275). Sequence comparisons were carried out with ClustalW software (http://www.ebi.ac.uk/Tools/clustalw2/index.html).

1.6.2. Tissue Collection and RNA Extraction

Expression analysis of the three GDSL sequences was carried out on wild type Ailsa craig plants in order to characterize various gene expressions: (i) in different tissues: fruit at 20 days after anthesis (DPA), inflorescence, anther, petal, sepal, young leaf, mature leaf, stem, hair, and root, (ii) during fruit development: 4, 8, 12, 20, 27, 35, 40 and 45 DPA, (iii) in separated fruit tissues: seed, locular tissue, columella, mesocarp and exocarp, at different stages of fruit development: 12, 20, 27 and 35 DPA. Another analysis compared GDSL expression profiles in cherry tomato between silencing and wild-type plants in leaves, flowers and fruits. For each tissue analyzed, the material collected was immediately frozen in liquid nitrogen and stored at −80° C. Total tomato RNA was isolated from leaf tissue using standard trizol RNA extraction protocol (Life Technologies, Basel, Switzerland) and further DNase treated (Turbo DNA-free Kit, Ambion/Applied Biosystems, Courtaboeuf, France). Four extractions were performed on each collected sample. RNA was separated by electrophoresis on a 1.2% agarose gel to check for degradation.

1.6.3. Quantification of Gene Expression Using qRT-PCR

One μg of DNase-treated total RNA was reverse transcribed as described by Lemaire-Chamley et al. (Lemaire-Chamley et al., 2005). The RT-product was diluted 10-fold in water, and aliquots of 2 μL were used in real time PCR reaction (25 iL final volume) in the presence of 0.2 μM clone-specific primers using the iQ™ SYBR Green Supermix (Bio-Rad, Marne La Coquette, France) according to the manufacturer's instruction. Real-time PCR was performed on a iCycler™ iQ (Bio-Rad) with the following parameters: a first step of denaturation at 95° C. for 3 min, followed by a 40 amplification cycles with denaturation step at 95° C. for 30 s, and an hybridation/synthesis step at 60° C. for 30 s. During last step, temperature increased of 60 to 97° C. with 0.5° C. amplification all 10 s in order to built fusion curve. Data acquisition and analysis was done using the iCycler™ iQ software (version 3.0a, Biorad). EiF4α (BT013166), b-tubulin (DQ205342) and actin were used as housekeeping genes allowing calculation of relative expression of interest genes: gdsl1, gdls2, gdsl3 using Genes (Gene Expression Analysis fro iCycler IQ Real Time PCR detection system) Excel applet from Bio-Rad. All specific primers used in quantitative RT-PCR are recapitulated in table II.

TABLE II
Specific primer sequences used for quantitative real time PCR

| gene | forward primer | reverse primer |
|---|---|---|
| gds11 | GCAACACTTCATGTTGTGCTA | CAATATCACACTCCACCCTAC |
| | CTT = SEQ ID NO: 37 | AAA = SEQ ID NO: 38 |
| gds12 | GCCAAATTCAACTGCGTCGTT | TCTCCTTCGCTACAACGTGGA |
| | T = SEQ ID NO: 39 | A = SEQ ID NO: 40 |
| gds13 | GGGTGGCCAATTACAAAATG = | ACTCACACACATCACATAAA |
| | SEQ ID NO: 41 | GATGC = SEQ ID NO: 42 |
| β-tubulin | AACCTCTCGTGGATCACAGC = | GGCAGAAGCTGTCAGGTAAC |
| | SEQ ID NO: 43 | G = SEQ ID NO: 44 |
| EiF4α | AGTGGACGATTTGGAAGGAA | GCTCCTCGATTACGACGTTG = |
| | G = SEQ ID NO: 45 | SEQ ID NO: 46 |
| actin | GGACTCTGGTGATGGTGTTAG = | CCGTTCAGCAGTAGTGGTG = |
| | SEQ ID NO: 47 | SEQ ID NO: 48 |

1.7. Subcellular Localization of GDSLJ

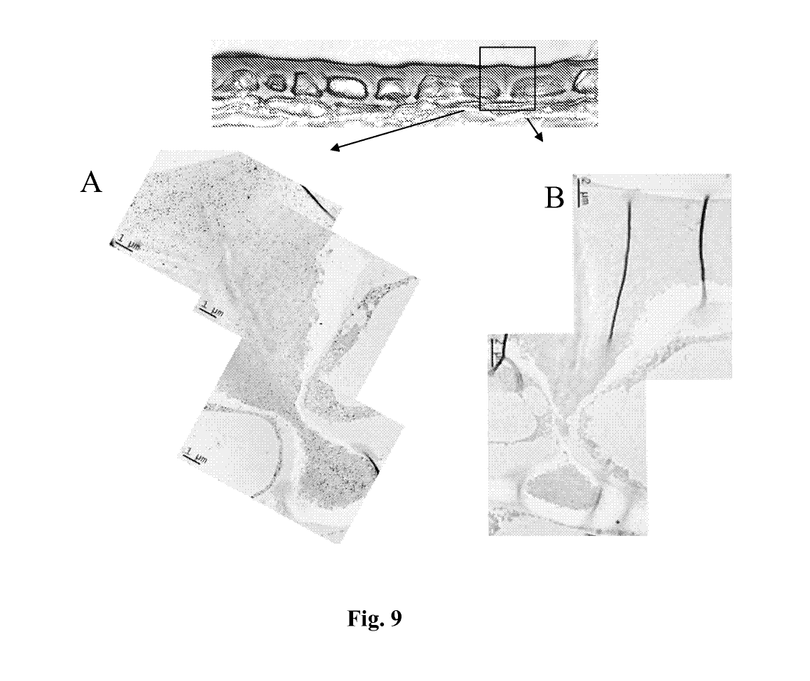

Thin ultramicrotome sections (100 nm) of Wt tomato exocarp were incubated in 3% (w/v) BSA in 10 mM PBS (pH 7.2) and incubated for 1 h with the polyclonal antibody diluted in 10 mM phosphate buffer saline supplemented with 1% BSA and 0.05% tween 20. After extensive washing, the section were incubated for 1 h with a gold-conjugated antibody (Aurion, NL). Labelling was intensified though the use of silver enhancement kit (Aurion, NL) according to the manufacturer instruction. After washing, the grids were stained with 2% uranyl acetate. Electrographs were taken with a Jeol 100S TEM electron microscope. (see FIG. 9)

1.8. Effect of RNAi on Plant

1.8.1. Analysis of Cutin Monomers

Cuticular waxes were twice extracted from isolated cuticles of tomato in 10 mL of methylene chloride. The resulting solution of cuticular waxes was spiked with 10 μg of tetracosane (Sigma) as internal standard. Extracted cutins were transesterified by treatment with 15% boron trifluoride in methanol (Sigma) for 16 h at 70° C. including 5 μg of heptadecanoic acid as internal standard. The organic products was extracted with CH₂Cl₂ (3×5 mL), dried over anhydrous sodium sulfate and dried under an N₂ stream. Prior to GC analysis, wax and cutin samples were sylilated with 50 μL of bis-(trimethylsilyl)trifluoroacetamide containing 1% trimethylchlorosilane (Sigma) at 70° C. for 60 min. The qualitative composition was studied with capillary GC-MS (Thermo trace GC ultra) equipped with (DB5ms, 30 m×0.32 mm, 0.1 mm [J&W]) and a mass spectrometric detector (Thermo DSQII, 70 eV, m/z 50-700). For cutin analyses, GC was carried out in an oven temperature programmed for 1 min at 90° C., 40° C.min-1 up to 200° C., 1 min at 200° C., 3° C.min-1 up to 320° C. For waxes analyses, GC was carried out in an oven temperature programmed for 2 min at 50° C., 40° C.min-1 up to 220° C., 2 min at 200° C., 3° C.min-1 up to 320° C., 30 min at 320° C. The quantitative composition was studied using GC-FID (Hewlett Packard 5890) under the same GC conditions, but H2 was used as carrier gas at 2 mL.min⁻¹.

1.8.2. FTIR Analysis.

FTIR was performed on a Nicolet Magna 550 ThermoElectron spectrometer (Madison, Wis.) equipped with a KBr beam splitter and a liquid-nitrogen-cooled mercurycadmium-telluride detector. Each spectrum is the result of the average of 200 scans in the 4000 to 600 cm⁻¹ range at 2 cm⁻¹ resolution apodized with a Happ-Genzel function. For ATR infrared spectroscopy, pieces (at least 0.5×0.5 cm) of cuticle and cutin (external and internal sides) were steadily spread on the surface of ZnSe or germanium crystals.

1.8.3. Toluidine Blue Staining

A toluidine blue assay was performed to identify defects in the permeability of fruit cuticles in wild type and transformed tomato plants (Tanaka et al., 2004; Hovav et al., 2007). Fruits were placed in cup filled with an aqueous solution of toluidine blue at 1% (Sigma, St Louis, Mo., USA). Approximately a quarter of the fruit surface was immersed for 4 h in the toluidine blue solution. Afterwards fruits were washed with distilled water to remove the excess of dye.

1.8.4. Light Microscopy and Cuticle Thickness of Transformed Plants

Slices of 20 DPA fruits cuticle from wt, 3.1.9, 10.10, 4.2, 17.3 plants were realized using a cryostat (Icrom HM5000M, Microm France). Samples were frozen and rolled up on it-self in cryoprotector. Cross-sections with a thickness of 20 μm were then obtained and observed with a light microscope (Leica DMRB, Leica) and a magnification of X20.

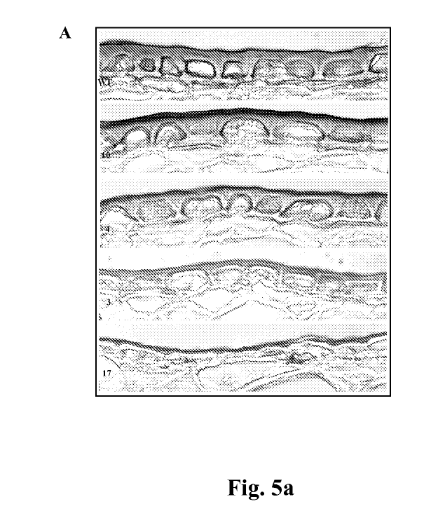

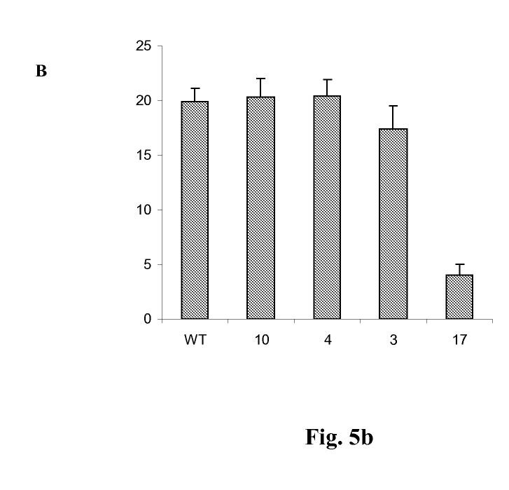

Cuticle thickness was measured from 3 different fruits (10 measures per fruit). For some transformant and wt plants (20 DPA and breaker +7 days), fruit exocarpe (including cuticule) were cut from two different fruits and immersed in 0.1 M phosphate buffer (pH 7.2) containing 3% paraformaldehyde and 0.1% glutaraldehyde for 16 h at 4° C. The samples were rinsed, dehydrated in ethanol series and embedded in LR white hard grade (Agar Aids). Ultramicrotom (Microm RMC MT 700) sections (1 μm) were obtained and were observed on a light microscope (Leica DMRB, Leica). Sections were examined on a LEICA DMRD. A band-pass filter 450-490 nm was used as excitation filters and primary fluorescence was detected at >515 nm. (see FIG. 5)

1.8.5. Atomic Force Microscopy

Cutin samples were fixed on a metallic support using double-side sticky tape. AFM height, phase and error-signal images were acquired in air using an Autoprobe CP Park Scientific Instrument (Sunnyvale, Calif.). AFM images were recorded in the Tapping mode using conventional pyramidal silicon nitride cantilevers obtained from Digital Instrument (Santa Barbara, Calif.). All the Tapping mode images were acquired at the lowest possible stable scanning force, less than 10 nN. Different surface areas were scanned from each sample 80 μm×80 μm, 40 μm×40 μm and 2 μm×2 μm). The mean surface roughness (RMS) parameter was deduced from the height images recorded with a surface of 2 μm×2 μm.

1.8.6. Scanning Electron Microscopy

The morphology of air dried cuticles extracted from the wild type and 17.3 plants were prepared as previously described (Bargel and Neinhuis, 2005) and were characterized by scanning electron microscopy (SEM). The samples were sputter coated with a fine gold layer (approx 100-200 A) before recording the SEM images using a Leo-1450VP scanning electron microscope (LEO Electron Microscopy Ltd, Cambridge, UK) and low-voltage conditions.

2. Results

2.1. Isolation, and Sequence Analysis of GDSL1 cDNA Clone

Preliminary proteomic study of tomato cutin allowed us to isolate an abundant GDSL-motif lipase-acylhydrolase called GDSL1 (TA37529_{—}4081, http://plantta.jcvi.org/search.shtml) from tomato cutin. The presence of this protein was confirmed by SDS-PAGE and immunoblotting of SDS extracts of cuticle powder using the specific antibodies produced against the corresponding recombinant protein. The cuticular localization of GDSL-motif lipase-acylhydrolase is in agreement with previous immunocytochemical studies on Agave Americana (Reina et al., 2007).

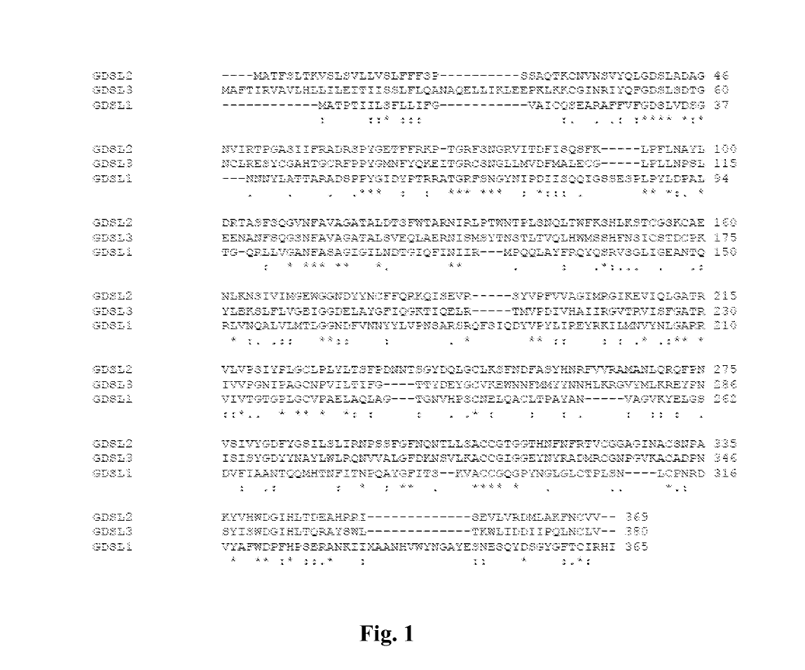

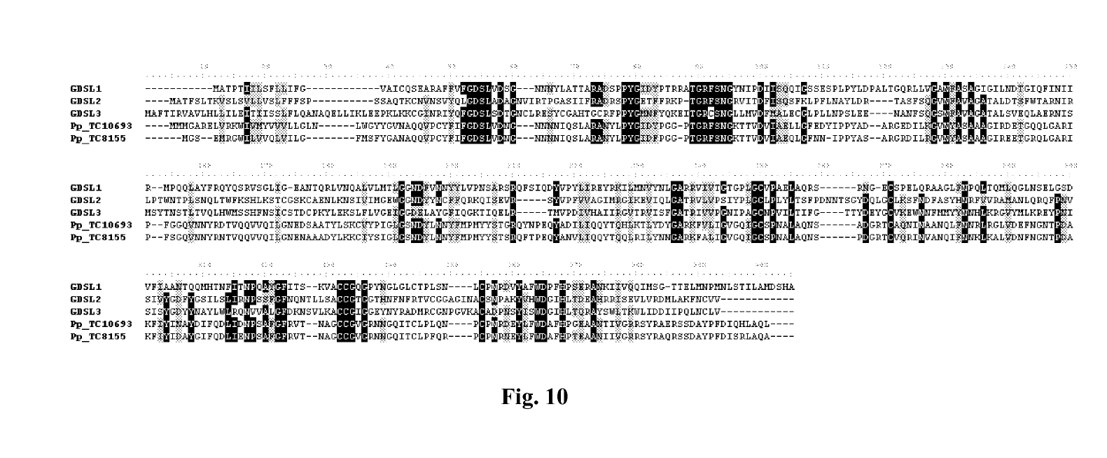

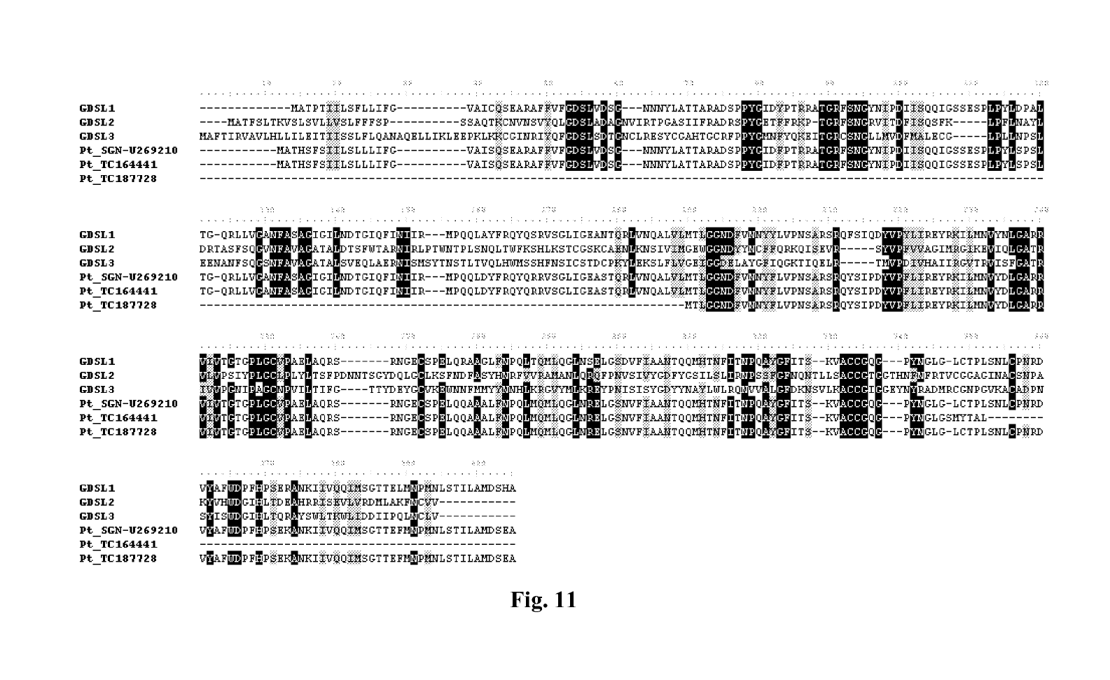

GDSL1 cDNA clone was then isolated from tomato exocarp by RT-PCR using specific primers derived from the nucleotidic sequence available on SGN tomato database (SGN-U313619, http://www.sgn.cornell.edu/). The full length cDNA (SEQ ID NO: 24, 1042 bp) contains an open reading frame coding a 362 amino acid protein (SEQ ID NO:27). GDSL1 precursor shows a theoretical pI of 7.55 and a theorical molecular mass of 39694.26 daltons. Peptide signal is predicted by SignalP software and cleavage site is situated between Cysl9 and Gln20. For mature GDSL1 protein, theoretical pI and molecular mass are respectively 7.75 and 37688 Da. Extracellular localisation of GDSL1 was predicted for GDSL1 by TargetP 1.1 software (http://www.cbs.dtu.dk/services/targetP). Extracellular localization seems to be a common feature of GDSL. Indeed, 107 putative gdsl coding genes have been recently listed (Ling, 2008) in Arabidopsis thaliana. Among the corresponding proteins, 99 were predicted as extracellular localized. GDSL-motif lipase-acylhydrolase proteins belong to a large multigenic family, characterized by a GDSL motif, five conserved blocks sequences (FIGS. 1, 10 and 11). Three amino-acids are described as typical catalytic triad for esterase and lipase activity (Ling et al., 2006): serine (S), aspartic acid (D) and histidine (H). In order to characterize GDSL lipase genes expressed during early tomato fruit development, we searched for proteins annotated as GDSL-motif lipase-acylhydrolase and/or presenting sequence homologies to GDSL1 in tomato SGN database. This screen allowed identifying 38 tomato unigenes.

Further selection was based on the presence of the full GDSL motif (20 remaining unigenes) and on the expression of these genes during early fruit developing (7 remaining unigenes). We focused our attention on three unigenes (SGN-U313619: GDSL1, SGNU315324:GDSL2, and SGN-U318275: GDSL3) since they were represented by more than one EST in early tomato fruit databases. Comparison of amino acid sequences (FIG. 1) reveals that GDSL1 shows 23 and 19.9% of identity with GDSL2 and GDSL3, respectively.

Alignment of amino acid sequences (FIG. 1) show conservation of GDSL lipase typical motif (glycine, aspartic acid, serine and leucine) between GDSLs. The SDH triad is also observed among tomato GDSLs. Serine of GDSL motif is present in bloc I, histidine residue in block V. As described in the literature, the aspartic acid residue is either localized in block III (Upton and Buckley, 1995) for GDSL1 and GDSL2 or in block V just upstream histidine (Akoh et al., 2004) for GDSL1, GDSL2 and GDSL3.

GDSL1 sequence was also compared to other lipases and hydrolases with GDSL motif of different plants presented in literature. GDSL1 shows moderate percentage of identity in amino acids with AgaSGNH of Agave americana (67.5%, AAS75127) (Reina et al., 2007) and LTL1 of Arabidopsis thaliana (64.9%, AAG51758) (Naranjo et al., 2006). Identity percentage is lower for other aligned plant proteins, CaGLIP1 of Capsicum annuum (33%. AAX20033) (Hong et al., 2008), CaGL1 of C. annuum (32.7%, AA223955) (Kim et al., 2008), GLIP1 of Arabidopsis thaliana (29.8%, NP_{—}198915) (Oh et al., 2005), ENOD8 of Medicago sativa (26.1%, AAB41547) (Pringle and Dickstein, 2004) and BnLIP2 of Brassica napus (21.5%, AAX59709) (Ling et al., 2006).

2.2. Spatio-Temporal Expression of Tomato GDSL1

2.2.1. GDSL Genes Show a Spatial-Specific Expression in Vegetative Tissues

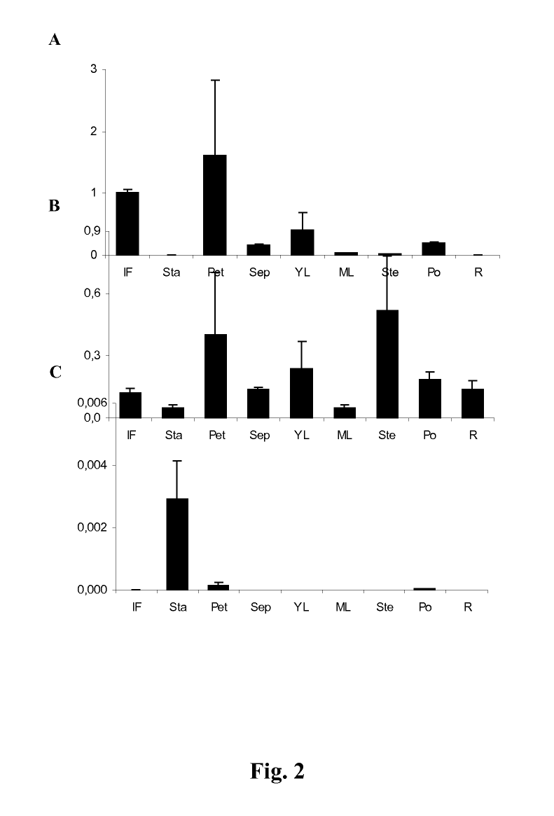

To investigate whether GDSL genes are expressed ubiquitously in tomato plants, quantitative RT-PCR was performed in various organs of wild-type plants: fruit at 20 DPA, inflorescences, anther, petal, sepal, young leaf, mature leaf, stem, hair, and root (FIG. 2). GDSL genes show important variation of expression levels. The mean transcript levels ranged in the order gdsl1>gds12 >gds13. Furthermore, each gdsl gene seems to be specifically expressed in one or several tissues and totally absent of others. GDSL1 transcripts are highly detected in petal, fruit, stem and young leaf, but not in anther and root (FIG. 2A). GDSL2 is expressed in all tissues and more especially in petal and stem (FIG. 2B). GDSL3 is slightly expressed and specifically in anther and petal (FIG. 2C).

2.2.2. GDSL Genes Show a Developmental Stage-Dependant Expression

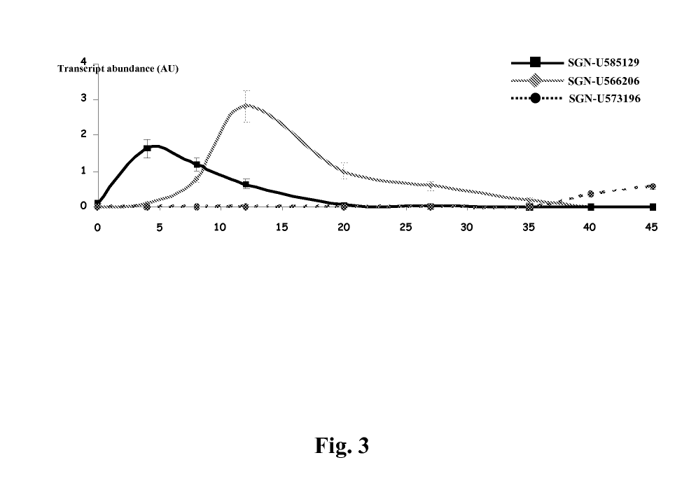

Furthermore, the temporal expression of GDSL was investigated during fruit development was performed in whole fruit. RNAs were purified from fruit samples collected at 4, 8, 12, 20, 27, 35, 40 and 45 DPA and used as template to detect GDSL transcript level by quantitative RT-PCR analysis (FIG. 3). GDSL1 and GDSL2 genes were expressed during early tomato development (4 to 35 DPA) whereas gds13 was preferentially expressed during fruit ripening (35-45 DPA). gdsl1 transcripts showed a peak of expression at 12 DPA and decreased slowly until the end of fruit development. The expression of GDSL2 decreased along fruit development whereas whose of gds13 increased at later stage. gdsl2 was the most precious: its highest level of expression was at 4DPA (days post anthesis) and decreased rapidly to nearly no expression as soon as 20DPA.

2.2.3. GDSL Genes Show a Tissue-Specific Expression in Tomato Fruit

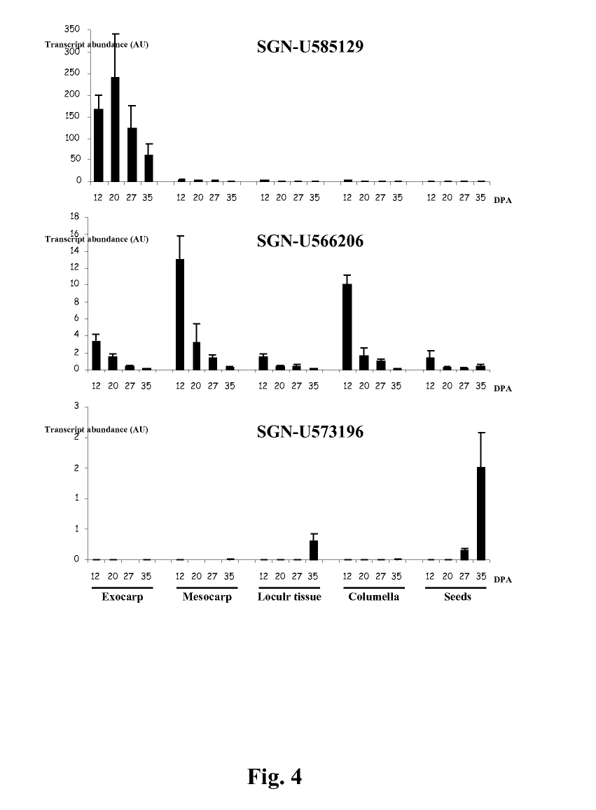

Finally, expression of GDSL genes in fruit during its development was investigated in 5 different fruit tissues: seed, locular tissue, columella, mesocarp and exocarp (FIG. 4). Along fruit development, gdsl genes also presented specific expression pattern

Each GDSL gene shows a proper expression pattern (FIG. 4). gdsl1 displays the highest expression level among the gdsl tested, as already observed in whole fruit.

Moreover, gdsl1 transcripts are specifically detected in exocarp. These transcripts are detected in this tissue at the four developmental stages tested, with a maximum of expression at 12 DPA. gdsl2 is expressed in all fruit tissues at early developmental stages. Its expression is more important in columella and exocarp at 12 DPA. gdsl3 transcripts are detected in seed and to a lower level in locular tissue at late developmental stages.

The enrichment of each tissue during this analysis revealed that GDSL1 highest expression is not at 12DPA as previously seen (FIG. 4) but more around 20DPA in exocarp. Expression rate reparation of GDSL2 is exocarp>columella>mesocarp>locular tissue>seeds and as expected, decreased during fruit development. Finally, SlGDSL3 expression is more important in seeds of 35DPA fruits, expression in locular tissue probably being a contamination by seeds during sampling.

2.3. Subcellular Immunolocalisation of GDSL1 Protein in Tomato Exocarp

To investigate GDSL1 subcellular localization and to confirm its secretion in extracellular matrix, immunolocalisation was conducted on 20 DPA wt tomato exocarp.

In electron micrographs realized on WT samples, the cuticle of exocarp was seen as an electron dense layer of 5.4 to 21 μm deposited over a less dense layer corresponding to cell walls as previously observed in Ailsa Craig tomato at red ripe stage (Saladié et al 2007). TEM analysis of cuticles from wt fruits showed that cuticle penetrates between epidermis cells, forming an anticlinal peg corresponding to the beginning of the outer cell wall cutinisation.

Immunogold electron microscopy revealed that GDSL1 was clearly localized in cuticle as well as in the underlying epidermis cells whereas no labelling was observed in cell wall layer. This result demonstrated that GDSL1 is excreted from the underlying cells to the extracellular cuticle.

2.4. Alteration of GDSL1 Lipase Expression by RNAi in Tomato

2.4.1. Generation of Gdsl1-RNAi Plants

Two RNAi constructions were generated, one using a 3′-UTR fragment and the other a 5′ fragment as inverted repeat under control of 35S promoter. Ten T0 plants were selected: 3.1, 3.6, 4, 10, 12, 14, 17, 18 and 20 for 5′-RNAi construct and 7.2 for 3′-RNAi construct. The analysis of gdsl1 gene expression allows to quantify gdsl1 gene extinction and to select the most interesting plants.

2.4.2. Modification of Gdsl Genes Expression in Gdsl1 Silenced Plants

To investigate gdsl1 extinction efficiency, the expression of gdsl1 in leaves and flowers of different silenced plants in comparison to wild-type plant was performed by quantitative RT-PCR. Different levels of expression decrease were observed in the transgenic plants. In leaves, all transformed plants presented a decrease in gdsl1 relative expression rate, except plant 3.6. And for plants 4, 14, 17 and 18 reduction raised 10 fold less than in wt leaves. In flowers, gdsl1 expression rate was strongly reduced in all transformed plants. 4, 12, 17, 18 and 20 transformed plants showed a relative gdsl expression rate 10 fold lower than the wt plant. Several plants seemed to be particularly affected with a residual expression level of gdsl1 of 10% in leaves and in flowers (4, 17 and 18).

In addition, the expression of gdsl2 and gdsl3 was measured in the same samples and showed an increase of expression rate of gdsl2 of several transformed plants in comparison of wild-type plant. In leaves, the gdsl2 expression was at least doubled in 3.1, 10 and 18 transformed plants and in flowers, gdsl2 presented an increased expression in plants 3.6, 7.2, 10, 12 and 18 (×1.5 in plants 3.6, 7.2, 10 and 12). These observations suggested a compensation of the decrease in gdsl1 expression by gdsl2. Nevertheless, no direct correlation was observed between the decrease of gdsl1 expression and the increase of gds12 transcript rate measured in some plants. For example, transformed plant 4 showed a decrease of gdsl1 expression rate in leaf but no increase of gdsl2 transcripts. On the contrary, the expression of gdsl1 in flowers was only slightly diminished in plant 3.6 and gdsl2 transcript rate was highly increased. No modification in expression rates of gds13 was observed.

2.4.3. Expression of Gdsl Lipases in Fruits

One phenotype observed on gdsl1 silenced plant was a modification of flower structure leading to a low fertilization rate in some transgenic lines (3.6, 7.2, 12, 18 and 20) resulting in the absence of fruits. Thus, expression rates of gdsl genes were only measured in wild-type plant and 3.1, 4, 10 and 17 transformed plants. An important reduction in gdsl1 expression rate was observed especially for plants 4 and 17, where the gdsl1 remaining expression was close to zero. Two and five fold decreases of gdsl1 expression were observed in fruits of transformed plants 3.1 and 10, respectively. In contrast with that was observed in leaves and flowers, in fruits, no increase of gdsl2 and gdsl3 expression rates was noticed. According to these observations, 3.1, 4, 10 and 17 transformed plants were chosen to produce T2 homozygous plants which were used for biophysic studies.

2.5. Morphological and Cuticular Defects Associated with the Loss of GDSL1 Function

2.5.1. Effects of Gdsl1 Gene Silencing on Plants

Influence of silencing of gdsl1 gene on cuticle structure was analyzed in T2 homozygous plants. Modifications of fruit colour at maturity and reduction of the number of fruits per truss was observed, but the major phenotype was the higher cuticle transparency and brightness on transformed plants. The use of hydrophilic toluidine blue dye (TB) coloration and the conservation tests allowed to suggest cuticle defects in 17.3 plants, and to less extend in 3.1.9 plants. In addition, tomato fruits were harvested at the red ripe growth stage of wild-type and transformed plants before any sign of dehydration and were stored at room temperature in order to assess their resistance to dehydration. After 3 weeks, only the 17.3 plant displayed visible sign of dehydration. Taken together, these results suggest that the fruit cuticle of the transformed plant is affected.

2.5.2. Determination of Cuticle Thickness in Transformed Plant

To investigate the influence of gdsl1 silencing on cuticle thickness, light microscopy observations were performed on 10 μm cross-sections of cuticle of mature fruits from T2 transformed and wild-type plant. As previously observed in red ripe tomato fruit (Bargel et al 2005; Buda et al 2009; Isaacson 2009) cuticles penetrates between epidermis cells, forming an anticlinal peg. Cuticle thickness was measured (i) at the cuticle membrane and (ii) between two epidermal cells including cuticle membrane and the anticlinal peg (FIG. 5).

Sharp differences were observed between Wt and the GDSL transformants particularly at the cuticle membrane level were the thickness reduction is correlated to the reduction of GDSL1 expression. (see FIG. 5)

2.5.3. Morphological Characterization of Cuticle and Cutin Surface and Cross Section

Cuticle surface of mature tomato cuticle from wt and 17.3 transformant plant were compared by SEM. In the case of wild type plant, cuticle exhibited a regular morphology as previously described in the literature (Hovav, 2007). By contrast, cuticle surface from 17.3 transformant plant seemed less regular and wrinkled. Cuticle and cutin surface morphology has also been investigated through SEM and AFM at 20 DPA. The SEM images showed a continuous cuticle displaying the imprint of the underlying epidermis cells. The impression of the underlying plant cell of the cuticle is more important for the 17.3 plant as the design of the corresponding cuticle cells are more apparent in the corresponding SEM images as compared with the wt fruit cuticle. Similar observation was obtained from the AFM images that have been performed from isolated dewaxed cuticle samples. As a consequence, the 17.3 phenotype can be attributed to a lower cutin thickness; which is not compensated by wax deposition. As previously observed (Round et al., 2000), wild type cutin surface displayed regular domains whereas cuticle from the 17.3 transformant showed a more irregular texture.

Furthermore, high resolution AFM images of the corresponding cutin surfaces (wt and 17.3) display notable differences in the surface microstructures. The RMS roughness value has been estimated to 10.4 nm and 7.4 nm for respectively the wild type and the 17.3 transformant. While the roughness values are quite similar, significant difference can be seen from the corresponding morphologies. The fine surface of the wild sample can be described as an assembly of regular small regions ranging from 20 to 80 nm whereas the surface of the 17.3 transformant is designed by larger and irregular nodules.

The co-ordinated use of AFM and SEM indicated surface differences that may be involved in the brightness differences observed between 17.3 and wt tomato fruits.

In addition, nanopores with a mean diameter of 20 nm were observed in the topography of the 17.3 cutin and were absent in the wild type cutin, whereas the same sample preparation procedure was used for both cutin. These surface features are confirmed in each case by the AFM height images, assuming identical recording conditions for both wild and 17.3 samples tomato fruit.

These nanopores could account for the differences in the dehydration rate observed between Wild type and 17.3 tomato fruit.

2.5.4. ATR FTIR

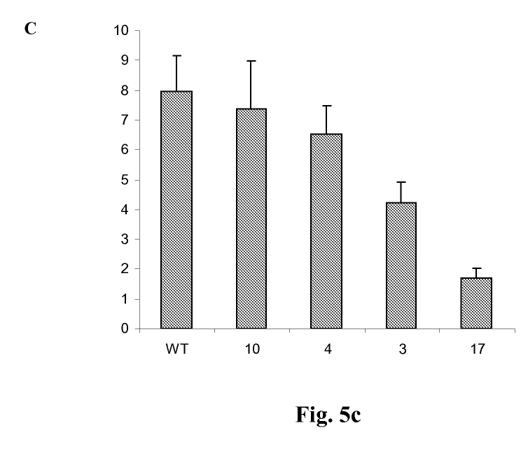

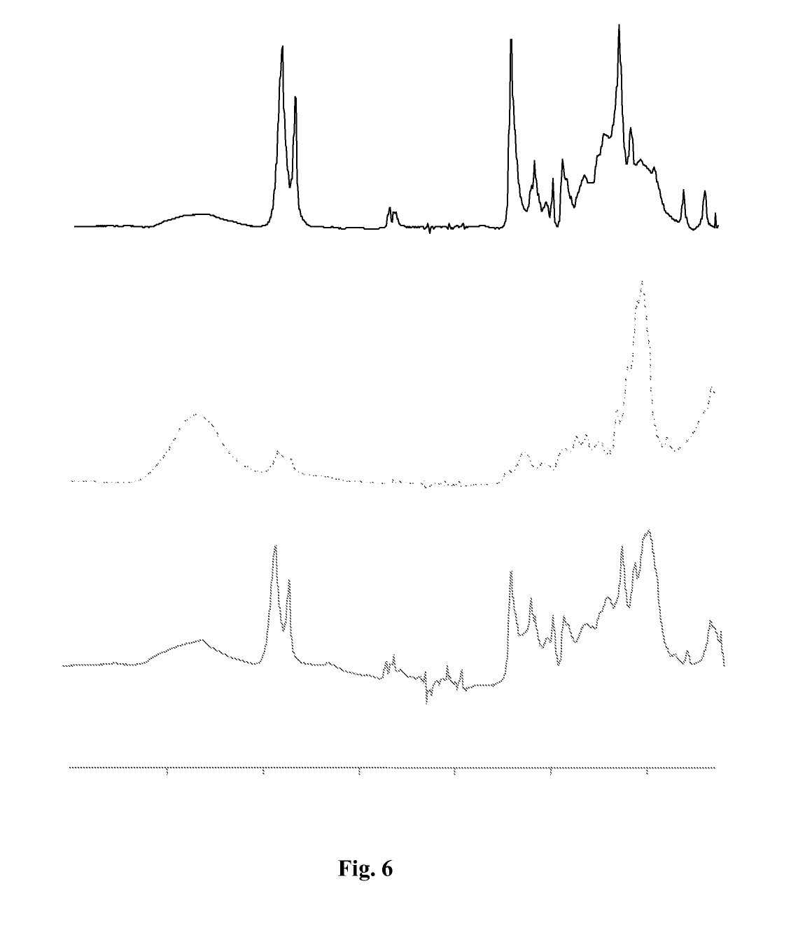

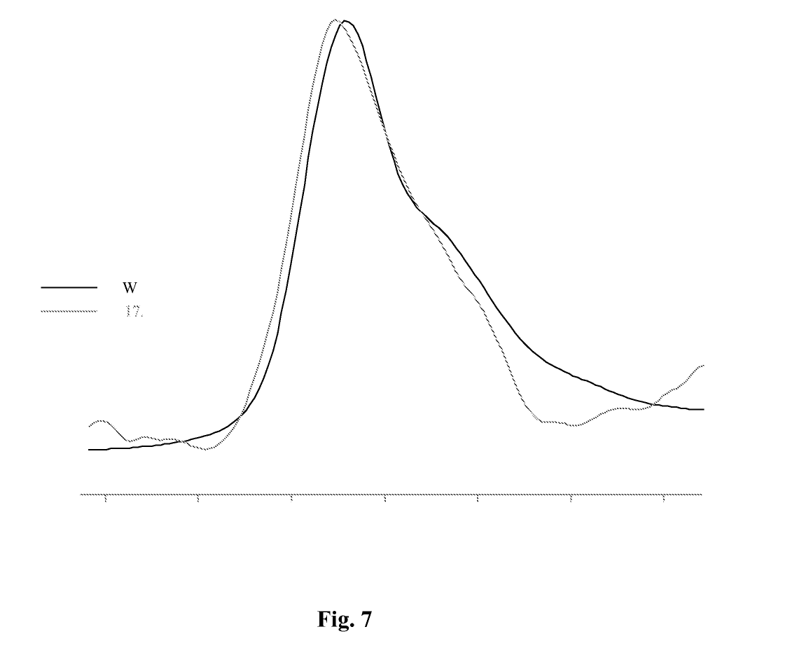

FTIR was performed on cuticle and cutin isolated from wild plant and T2 transformant plants (4.2, 3.1.9, 10.10, 17.3). ATR spectroscopy is an effective technique for studying biological material because sample can be oriented on the ATR crystal. As a matter of fact, FTIR spectra were obtained from the external side and from the internal or “cell wall” side of cuticle (FIGS. 6 and 7). From the external side, the spectra display typical cutin spectra dominated by aliphatic CH2 asymmetric and symmetric stretching bands at 2919 cm-1 and 2850 cm-1. Cutin show also a major band at 1729 cm-1 with a shoulder at 1708 cm-1 assigned to C═O stretching vibration of ester groups. The shoulder at 1708 cm-1 indicates that some of these functional group are involved in hydrogen bounding (Chen et al., 2005). The band at 1167 cm-1 is attributed to C—O ester bound. By contrast, spectra from the internal side of the cutin are dominated by a major polysaccharide feature at 1032 cm-1 (da Luz, 2006) and a characteristic broad OH bands in the 3360 cm-1 range. The obtained spectra were very similar between all plants except for the 17.3 transformant were the polysaccharide features are more important (as compared to the C═O moieties) than in the wild type. This may be explained as a result of a thinner cutin layer. According to the C═O stretching vibration, the same shoulder at 1708 cm-1 was observed for all plants, which suggest that the hydrogen bounding in the cuticle are not modified through gdsl1 silencing although a slight shift (2cm-1) was observed in the case of 17.3 plant.

2.5.5. Cutin Composition

The cutin fraction isolated density from the different transformant and wild-type plant at the red ripe stage was 0.418 ±0.08 mg/cm-2 for 17.3, 0.856 ±0.002 mg/cm-2 for 3.1.9, 1.589 ±0.21 mg/cm-2 for 4.2 and 1.03 ±0.13 mg/cm-2 for 10.10 transformed plants in comparison to 1,336±0.24 mg/cm-2 for wt plant. According to the literature (Baker et al., 1982; Osman et al., 1995) tomato cutin comprises 16-hydroxyhexadecanoic acid and dihydroxyhexadecanoic acids. It must be noticed however, that a significant amount of dicarboxylic acids, usually described as suberin monomers were also extracted from tomato cuticle. A similar result was reported from red tomato (Wang et al., 2000). Data are the average of three replications and variability for the cutin composition was high.

Nevertheless, according to this study, there were no significant differences in the relative composition (%) of cutin constituents for the different tested plants, except for a smaller (p<0.05) proportion of 16-hydroxyhexadecanoic acid.

2.6. In Vitro Activity of GDSL1 Recombinant

2.6.1. Transformed Escherichia Coli Express Hydrolase Activity on Tributyrin Agar

Observation of tributyrin agar plates after 48 h incubation gives a rapid obviousness of hydrolytic activity of bacterial culture. The diffusion test in tributyrin agar showed clear precipitate zones for E. coli expressed Thr-GDSL1 but not in E. coli transformed by pETD vector without insert. The depth of clear precipitate zone around wells increases during incubation time.

2.6.2. Recombinant GDSL1 (rGDSL1) Show Esterase Activity Toward Substrates with Short Chain Lengths

Hydrolytic activities of rGDSL1 and Thr-GDSL1 were measured using various pnitrophenyl derivatives with different acyl group chain lengths: p-nitrophenyl acetate (C2), p-nitrophenyl butyrate (C4), p-nitrophenyl laurate (C12), p-nitrophenyl myristolate (C14), and p-nitrophenyl palmitate (C16). Chain length of acyl groups allow discriminate esterase and lipase activities of hydrolytic enzymes. Enzymes shown lipase activity were characterized by their ability to hydrolyze a wide range of fatty acid esters whereas enzymes with esterase activity hydrolyze preferentially short chain fatty acid esters (Pringle and Dickstein, 2004; Hong et al., 2008). We found a hydrolase activity for rGLDS1 and Thr-GDSL1 using p-nitrophenyl acetate (C2), p-nitrophenyl butyrate (C4). No significant hydrolytic activity is detected when longer chains of pnitrophenyl esters were used as substrate.

3. Discussion

GDSL-motif lipase-acylhydrolase forms a large multigenic family and in the smallest plant genome sequenced, Arabidopsis thaliana 107 non redundant members have been recovered from data mining (Ling, 2008). The tetrapeptide motif G-D-S-L, the signature of this family of proteins is generally located close to the N-terminal region of the amino acid sequence (Akoh et al., 2004; Ling, 2008). The serine residue contained in this motif is involved in the hydrolase activity and differs from common lipases where the catalytic serine residue belong to a GxSxG motif located in the middle regions of their amino acid sequences (Akoh et al., 2004). No tri-dimensional structure is available for a plant member of this protein family. Only the crystal structure of a bacterial GDSL esterase has been solved and has been used as template for determining by homology the residues involved in the hydrolase activity and to highlight the consensus polypeptide blocks that contain the residues involved in catalysis and binding of substrates (Lo et al., 2003).

Interestingly, this unique structure showed that GDSL-motif lipase-acylhydrolases belong to the large family α/β hydrolase (Lo et al., 2003). Here, the GDSL lipase isolated and characterized from tomato fruit belongs obviously to this protein family and the different conserved blocks can be highlighted in its amino acid sequence. Although these proteins become more and more attractive in the recent years, their function is still unknown. The GDSL-motif lipase-acylhydrolases characterized so far have been related to plant defense (JPN1 (Kram et al., 2008), CaGL1 (Kim et al., 2008), Glip1 (Oh et al., 2005), AtLTL1 (Naranjo et al., 2006)), germination (Ling et al., 2006; Clauss et al., 2008) and morphogenesis (Ling et al., 2006). In the present study, it was clearly demonstrated that GDSL-motif lipase-acylhydrolases are also involved in the formation of plant cuticle.

All the data obtained show that the GDSL-motif lipase-acylhydrolase, GDSL1, is involved in the formation of the cutin polyester. Several lines of evidence support these hypotheses.

First, GDSL was an abundant protein in mature tomato cuticle whereas gdsl1 expression reaches its maximum at 12 DPA. Moreover, GDSL1 was secreted in the cell wall apoplast as verified by expression of YFP-GDSL1 fusion protein and in agreement with the presence of a signal peptide at the N-terminus. Extracellular localization is quite a common feature of the GDSLs described so far (Oh et al., 2005; Hong et al., 2008; Kim et al., 2008). Finally (iii) in tomato fruit, 32 unigenes expressed in fruit are presented in databank (SOL genomics network). Numerous copies of the same family genes suggest an important role in plant development. A high efficiency in gdsl1 silencing demonstrates its importance in cutin formation. Nevertheless, silencing of gdsl1 expression might be compensated by the expression of other GDSL genes. For instance, even if its expression level is low, gdsl2 is expressed in the exocarp at the early stage of fruit development. As a consequence, GDSL2 production might be sufficient to maintain a minimal cutin formation along the fruit development.

With regard to the function of GDSL1, in vitro analysis of the recombinant protein revealed an esterase activity. Esterase activity has also been described for other GDSLs.Enod8, a GDSL isolated from Medicago sativa nodules (Pringle and Dickstein, 2004) and associated to suberin formation exhibited in vitro esterase activity. Recently, BnSCE3 isolated from Brassica napus and involved in the early stage of seed germination showed a sinapine esterase activity (Clauss et al., 2008).

On the contrary, delineation of the cuticular phenotype of transformed plant suggests that the protein is closely related to the polymerization of the cutin. A double function including both esterase/hydrolase and transferase activities has already been demonstrated for other lipases like lipolytic enzyme of Aeromonas hydrophile (Upton and Buckley, 1995).

The GDSL transformant exhibited a sharp reduction of cuticle thickness. In addition, severe deformation of epidermal cell wall was not observed. These results contrast with the cuticle of BODYGUARD mutants. Bodyguard encodes a protein candidate for cutin polymerization and/or anchoring the lipid polymers to the cell wall. Actually, Kurduyov and coworkers (Kurdyukov et al., 2006) observed an irregular cutin accumulation (above and within the cell wall) associated with the accumulation of cutin and wax in the Arabidopsis plants interrupted in the BODYGUARD gene. Interestingly, BODYGUARD encodes also an esterase with the α/β hydrolase fold. However, in regard to the GDSL-motif lipase-acylhydrolases, this esterase belongs to a different family of proteins. As a consequence, GDSL-motif lipase-acylhydrolases and α/β hydrolases, the two candidates for cutin polymerization are differently regulated and are involved in different mecanisms.

### REFERENCES

Akoh, C. C., Lee, G.-C., Liaw, Y.-C., Huang, T.-H., and Shaw, J.-F. (2004). GDSL family of serine esterases/lipases. Progress in Lipid Research 43, 534-552.
Baker, E. A., Bukova C, M. J., and Hunt, G. M. (1982). Composition of tomato fruit cuticle as related to fruit growth and development. In The plant cuticle, D. F. Cutler, K. L. Alvin, and C. E. Price, eds (New York: academic press), pp. 33-44.
Bargel, H., and Neinhuis, C. (2005). Tomato (Lycopersicon esculentum Mill.) fruit growth and ripening as related to the biomechanical properties of fruit skin and isolated cuticle. Journal of Experimental Botany 56, 1049-1060.
Beisson, F., Li, Y. H., Bonaventure, G., Pollard, M., and Ohlrogge, J. B. (2007). The acyltransferase GPAT5 is required for the synthesis of suberin in seed coat and root of Arabidopsis. The Plant Cell 19, 351-368.
Bessire, M., Chassot, C., Jacquat, A.-C., Humphry, M., Borel, S., MacDonald-Comber Petétot, J., Métraux, J.-P., and Nawrath, C. (2007). A permeable cuticle in Arabidopsis leads to a strong resistance to Botrytis cinerea. Embo Journal 26, 2158-2168.
Bird, D. A. (2008). The role of ABC transporters in cuticular lipid secretion. Plant Science 174, 563-569.
Blee, E., and Schuber, F. (1993). Biosynthesis of cutin monomers: involvement of a lipoxygenase/peroxygenase pathway. The Plant Journal 4.
Chen, B., Zhu, L., Zhu, J., and Xing, B. (2005). Configurations of the Bentonite-Sorbed Myristylpyridinium Cation and Their Influences on the Uptake of Organic Compounds. Environmental Science and Technology 39, 6093-6100.
Clauss, K., Baumert, A., Nimtz, M., Milkowski, C., and Dieter Strack. (2008). Role of a GDSL lipase-like protein as sinapine esterase in Brassicaceae. The Plant Journal 53, 802-813.
da Luz, B. R. (2006). Attenuated total reflectance spectroscopy of plant leaves: a tool for ecological and botanical studies. New Phytologist 172, 305-318.
Gilbert, R. D., Johnson, A. M., and Dean, R. A. (1996). Chemical signals responsible for appressorium formation in the rice blast fungus Magnaporthe grisea. Physiological and Molecular Plant Pathology 48, 335-346.
Graça, J., Schreiber, L., Rodrigues, J., and Pereira, H. (2002). Glycerol and glyceryl esters of omega-hydroxyacids in cutins. Phytochemistry 61, 205-215.
Hamza, S., and Chupeau, Y. (1993). Re-evaluation of Conditions for Plant Regeneration and Agrobacterium-Mediated Transformation from Tomato (Lycopersicon esculentum). Journal of Experimental Botany 44, 1837-1845.
Hong, J., Choi, H., Hwang, I., Kim, D., Kim, N., Choi, D., Kim, Y., and Hwang, B. (2008). Function of a novel GDSL-type pepper lipase gene, CaGLIP1, in disease susceptibility and abiotic stress tolerance. Planta 227, 539-558.
Hovav, R., Chehanovsky, N., Moy, M., Jetter, R., and Schaffer, A. A. (2007). The identification of a gene (Cwp1), silenced during Solanum evolution, which causes cuticle microfissuring and dehydration when expressed in tomato fruit. The Plant Journal 52, 627-639.
Iwamoto, M., Takeuchi, Y., Takada, Y., and Yamaoka, N. (2002). Coleoptile surface cuticle of barley is involved in survival and penetration of Blumeria graminis. Physiological and Molecular Plant Pathology 60, 31-38.
Kannangara, R., Branigan, C., Liu, Y., Penfield, T., Rao, V., Mouille, G., Hofte, H., Pauly, M., Riechmann, J. L., and Broun, P. (2007). The Transcription Factor WIN1/SHN1 Regulates Cutin Biosynthesis in Arabidopsis thaliana. The Plant Cell 19, 1278-1294.
Karimi, M., Inzé, D., and Depicker, A. (2002). GATEWAY™ vectors for Agrobacteriummediated plant transformation. Trends in Plant Science 7, 193-195.
Kersteins, G. (1996). Plant cuticles: an integrated approach. (oxford: BIOS Scientific Publisherd Limited).
Kim, K.-J., Lim, J. H., Kim, M. J., Kim, T., Chung, H. M., and Paek, K.-H. (2008). GDSLIipase1 (CaGL1) contributes to wound stress resistance by modulation of CaPR-4 expression in hot pepper. Biochemical and Biophysical Research Communications 374, 693-698.
Kolattuduky, P. (1996). Biosynthetic pathways of cutin and waxes, and their sensitivity to environmental stresses. In Plant cuticles: an integrated approach, G. Kersteins, ed (Oxford: BIOS Scientific Publishers Limited), pp. 83-118.
Kram, B., Bainbridge, E., Perera, M., and Carter, C. (2008). Identification, cloning and characterization of a GDSL lipase secreted into the nectar of Jacaranda mimosifolia. Plant Molecular Biology 68, 173-183.
Kurdyukov, S., Faust, A., Nawrath, C., Bar, S., Voisin, D., Efremova, N., Franke, R., Schreiber, L., Saedler, H., Metraux, J.-P., and Yephremov, A. (2006). The Epidermis-Specific Extracellular BODYGUARD Controls Cuticle Development and Morphogenesis in Arabidopsis. The Plant Cell 18, 321-339.
Lemaire-Chamley, M., Petit, J., Garcia, V., Just, D., Baldet, P., Germain, V., Fagard, M., Mouassite, M., Cheniclet, C., and Rothan, C. (2005). Changes in Transcriptional Profiles Are Associated with Early Fruit Tissue Specialization in Tomato. Plant Physiology 139, 750-769.
Li, Y., Fred, B., Abraham, J. K. K., Isabel, M., Mike, P., and John, O. (2007). Identification of acyltransferases required for cutin biosynthesis and production of cutin with suberin-like monomers. Proceedings of the National Academy of Sciences of the USA 104, 18339-18344.
Ling, H. (2008). Sequence analysis of GDSL lipase gene family in Arabidopsis thaliana. Pakistan Journal of Biological Sciences 11, 763-767.
Ling, H., Zhao, J., Zuo, K., Qiu, C., Yao, H., Qin, J., Sun, X., and Tang, K. (2006). Isolation and expression analysis of a GDSL-like lipase gene from Brassica napus L. Journal of Biochemistry and Molecular Biology 39, 297-303.
Lo, Y.-C., Lin, S.-C., Shaw, J.-F., and Liaw, Y.-C. (2003). Crystal Structure of Escherichia coli Thioesterase Protease Lysophospholipase L1: Consensus Sequence Blocks Constitute the Catalytic Center of SGNH-hydrolases through a Conserved Hydrogen Bond Network. Journal of Molecular Biology 330, 539-551.
Naranjo, M. A., Forment, J., Roldan, M., Serrano, R., and Vicente, O. (2006). Overexpression of Arabidopsis thaliana LTL1, a salt-induced gene encoding a GDSLmotif lipase, increases salt tolerance in yeast and transgenic plants. Plant, Cell and Environment 29, 1890-1900.
Nawrath, C. (2006). Unraveling the complex network of cuticular structure and function. Current Opinion in Plant Biology Physiology and metabolism 9, 281-287.
Oh, I., Park, A., Bae, M., Kwon, S., Kim, Y., Lee, J., Kang, N., Lee, S., Cheong, H., and Park, O. K. (2005). Secretome analysis reveals an Arabidopsis lipase involved in defense against Alternaria brassicicola. The Plant Cell 17, 2832-2847.
Osman, S. F., Gerard, H. C., Fett, W. F., Moreau, R. A., and Dudley, R. L. (1995). Method for the production and characterization of tomato cutin oligomers. Journal of Agricultural and Food Chemistry 43, 2134-2137.
Panikashvili, D., Savaldi-Goldstein, S., Mandel, T., Yifhar, T., Franke, R. B., Hofer, R., Schreiber, L., Chory, J., and Aharoni, A. (2007). The Arabidopsis DESPERADO/AtWBC11 Transporter Is Required for Cutin and Wax Secretion. Plant Physiology 145, 1345-1360.
Petracek, P. D., and Bukovac, M. J. (1995). Rheological Properties of Enzymatically Isolated Tomato Fruit Cuticle. Plant Physiology 109, 675-679.
Pollard, M., Beisson, F., Li, Y., and Ohlrogge, J. B. (2008). Building lipid barriers: biosynthesis of cutin and suberin. Trends in Plant Science 13, 236-246.
Pringle, D., and Dickstein, R. (2004). Purification of ENOD8 proteins from Medicago sativa root nodules and their characterization as esterases. Plant Physiology and Biochemistry 42, 73-79.
Pyee, J., Yu, H. S., and Kolattukudy, P. E. (1994). Identification of a Lipid Transfer Protein as the Major Protein in the Surface Wax of Broccoli (Brassica oleracea) Leaves. Archives of Biochemistry and Biophysics 311, 460-468.
Rashid, N., Shimada, Y., Ezaki, S., Atomi, H., and Imanaka, T. (2001). Low-Temperature Lipase from Psychrotrophic Pseudomonas sp. Strain KB700A. Applied and Environmental Microbiology 67, 4064-4069.
Reina, J. J., Guerrero, C., and Heredia, A. (2007). Isolation, characterization, and localization of AgaSGNH cDNA: a new SGNH-motif plant hydrolase specific to Agave americana L. leaf epidermis. Journal of Experimental Botany 58, 2717-2731.
Riederer, M., and Schreiber, L. (2001). Protecting against water loss: analysis of the barrier properties of plant cuticles. Journal of Experimental Botany 52, 2023-2032.
Round, A. N., Yan, B., Dang, S., Estephan, R., Stark, R. E., and Batteas, J. D. (2000). The Influence of Water on the Nanomechanical Behavior of the Plant Biopolyester Cutin as Studied by AFM and Solid-State NMR. Biophysical Journal 79, 2761-2767.
Ruiz, C., Falcocchio, S., Xoxi, E., Javier Pastor, F. I., Diaz, P., and Saso, L. (2004). Activation and inhibition of Candida rugosa and Bacillus-related lipases by saturated fatty acids, evaluated by a new colorimetric microassay. Biochimica and Biophysica Acta (BBA)—General Subjects 1672, 184-191.
Schnurr, J., Shockey, J., and Browse, J. (2004). The Acyl-CoA Synthetase Encoded by LACS2 Is Essential for Normal Cuticle Development in Arabidopsis. The Plant Cell 16, 629-642.
Schweizer, P., Felix, G., Buchala, A., Muller, C., and Metraux, J. P. (1996). Perception of free cutin monomers by plant cells. The Plant Journal 10, 331-341.
Shieh, M. W., Wessler, S. R., and Raikhel, N. V. (1993). Nuclear Targeting of the Maize R Protein Requires Two Nuclear Localization Sequences. Plant Physiology 101, 353-361.
Sieber, P., Schorderet, M., Ryser, U., Buchala, A., Kolattukudy, P., Metraux, J.-P., and Nawrath, C. (2000). Transgenic Arabidopsis Plants Expressing a Fungal Cutinase Show Alterations in the Structure and Properties of the Cuticle and Postgenital Organ Fusions. The Plant Cell 12, 721-738.
Sterk, P., Booij, H., Schellekens, G. A., Van Kammen, A., and De Vries, S. C. (1991). Cell-specific expression of the carrot EP2 lipid transfer protein gene. The Plant Cell 3, 907-921.
Suh, M. C., Samuels, A. L., Jetter, R., Kunst, L., Pollard, M., Ohlrogge, J., and Beisson, F. (2005). Cuticular Lipid Composition, Surface Structure, and Gene Expression in Arabidopsis Stem Epidermis. Plant Physiology 139, 1649-1665.
Taketa, S., Amano, S., Tsujino, Y., Sato, T., Saisho, D., Kakeda, K., Nomura, M., Suzuki, T., Matsumoto, T., Sato, K., Kanamori, H., Kawasaki, S., and Takeda, K. (2008). Barley grain with adhering hulls is controlled by an ERF family transcription factor gene regulating a lipid biosynthesis pathway. Proceedings of the National Academy of Sciences of the USA 105, 4062-4067.
Tanaka, T., Tanaka, H., Machida, C., Watanabe, M., and Machida, Y. (2004). A new method for rapid visualization of defects in leaf cuticle reveals five intrinsic patterns of surface defects in Arabidopsis. The Plant Journal 37, 139-146.
Upton, C., and Buckley, J. T. (1995). A new family of lipolytic enzymes? Trends in Biochemical Sciences 20, 178-179.
Waltson, T. (1990). Waxes, cutin and suberin. In Lipids, membranes and aspects of photobiology, J. Harwood and J. Boyer, eds (London: Academic Press), pp. 105-158.
Wang, C., Chin, C., and Gianfagna, T. (2000). Relationship between cutin monomers and tomato resistance to powdery mildew infection. Physiological and Molecular Plant Pathology 57.
Wellesen, K., Durst, F., Pinot, F., Benveniste, I., Nettesheim, K., Wisman, E., Steiner-Lange, S., Saedler, H., and Yephremov, A. (2001). Functional analysis of the LACERATA gene of Arabidopsis provides evidence for different roles of fatty acid omega-hydroxylation in development. Proceedings of the National Academy of Sciences of the USA 98, 9694-9699.
Xiao, F., Goodwin, S. M., Xiao, Y., Sun, Z., Baker, D., Tang, X., Jenks, M. A., and Zhou, J. M. (2004). Arabidopsis CYP86A2 represses Pseudomonas syringae type III genes and is required for cuticle development. Embo Journal 23, 2903-2913.
McCallum et al, 2000 Nat Biotechnol. 18(4):455-7; reviewed by Stemple, 2004, Nat Rev Genet. 5(2): 145-50.
Colbert et al., 2001, Plant Physiol. 126(2):480-4
Slade et al., 2005, Nat Biotechnol. 23(1):75-81
Lightner and Caspar, 1998, “Seed mutagenesis of Arabidopsis”, In: Methods in Molecular Biology: Arabidopsis Protocols (eds. J. M. Martinez-Zapater and J. Salinas), pp. 91-103. Totowa, N.J.: Humana Press)
Draper et al., 2004, Methods Cell Biol. 2004;77:91-I12
Buda G J, Isaacson T, Matas A J, Paolillo D J, Rose J K. Three-dimensional imaging of plant cuticle architecture using confocal scanning laser microscopy.
Isaacson T, Kosma D K, Matas A J, Buda G J, He Y, Yu B, Pravitasari A, Batteas J D, Stark R E, Jenks M A, Rose J K. Cutin deficiency in the tomato fruit cuticle consistently affects resistance to microbial infection and biomechanical properties, but not trans ational water loss.

1.-22. (canceled)

23. An isolated plant GDSL-lipase polypeptide differentially expressed between the different steps of the plant development, the different tissues of the plant, or both comprising the sequences SEQ ID NO:1, SEQ N0:2, SEQ ID N0:3, SEQ ID N0:4 and SEQ ID N0:5 in this order from the N-terminal to the C-terminal ends.

24. The isolated plant GDSL-lipase polypeptide according to claim 23, which is differentially expressed between the different steps of fruit development, the different tissues of the fruit, or both.

25. The isolated plant GDSL-lipase polypeptide according to claim 23, which is differentially expressed between seed, locular tissue, columella, mesocarp and exocarp of the fruit of the plant.

26. The isolated plant GDSL-lipase polypeptide according to claim 23, which is specifically expressed in the exocarp of the fruit of the plant.

27. The isolated plant GDSL-lipase polypeptide according to claim 23 comprising the sequences SEQ ID NO:1, SEQ N0:2, SEQ ID N0:3, SEQ ID N0:4 and SEQ ID N0:5 in this order from the N-terminal to the C-terminal ends separated by sequences which present at least 60% identity with the sequences of SEQ ID N0:6, SEQ ID N0:7, SEQ ID N0:8, orthologs thereof or derivatives thereof.

28. The isolated plant GDSL-lipase polypeptide according to claim 23 comprising the sequences SEQ ID N0:1, SEQ N0:2, SEQ ID N0:3, SEQ ID N0:4 and SEQ ID N0:5 in this order from the N-terminal to the C-terminal ends separated by sequences which present at least 90% identity with the sequences of SEQ ID N0:6, SEQ ID N0:7, SEQ ID N0:8, orthologs thereof or derivatives thereof.

29. An isolated plant GDSL-lipase polypeptide according to claim 23 comprising
(a) a polypeptide sequence selected from the group consisting of SEQ ID N0:6, SEQ IDNO:7 and SEQ ID N0:8;
(b) a polypeptide sequence which is at least 60% identical to the polypeptide of (a), wherein said polypeptide is differentially expressed between the different steps of the plant development, the different tissues of the plant or both;
(c) a polypeptide sequence which is at least 90% identical to the polypeptide of (a), wherein said polypeptide is differentially expressed between the different steps of the plant development, the different tissues of the plant or both; or
(d) a polypeptide sequence being a fragment of any one of (a) to (c), wherein said fragment or biologically active portion thereof is differentially expressed between the different steps of the plant development, the different tissues of the plant or both.

30. The isolated plant GDSL-lipase polypeptide according to claim 29, which is differentially expressed between the different steps of fruit development, the different tissues of the fruit, or both.

31. An isolated plant GDSL-lipase polypeptide according to claim 23 comprising the sequence SEQ ID N0:6, SEQ ID N0:7or SEQ ID N0:8.

32. An isolated plant GDSL-lipase polypeptide according to claim 23, wherein said sequence is a tomato sequence, a hot pepper sequence or a sweet pepper sequence.

33. An isolated polynucleotide comprising a nucleic acid molecule encoding for a polypeptide of claim 23.

34. The polynucleotide of claim 33, comprising a sequence selected from SEQ ID N0:9, SEQ ID N0:10 or SEQ ID N0:11.

35. A vector comprising the polynucleotide of claim 33 which polynucleotide optionally comprises a sequence selected from SEQ ID N0:9, SEQ ID N0:10 or SEQ ID N0:11.

36. A host cell genetically engineered with the polynucleotide of claim 33 which polynucleotide optionally comprises a sequence selected from SEQ ID N0:9, SEQ ID NO:10 or SEQ ID NO:11.

37. A host cell genetically engineered with the vector of claim 35.

38. A monoclonal antibody directed to a polypeptide as claimed in claim 1.

39. A plant or part of plant genetically engineered to not express at least partially an isolated plant GDSL-lipase polypeptide according to claim 1.

40. A tomato plant or part thereof genetically engineered to not express at least partially an isolated plant GDSL-lipase polypeptide selected from GDSL1 (SEQ ID N0:6), GDSL2 (SEQ ID N0:7) or GDSL3 (SEQ ID N0:8).

41. The tomato plant or part thereof of claim 40 which is a tomato fruit and which is genetically engineered to not express at least partially GDSL1 (SEQ ID N0:6).

42. A plant or part thereof genetically engineered to express an isolated plant GDSL-lipase polypeptide according to claim 23 having an abnormal activity.

43. The plant, plant part or fruit according to claim 39, which is transgenic.

44. The plant, plant part or fruit of claim 43 which is a tomato plant, tomato plant part or tomato fruit.

45. A plant, plant part or fruit which is transgenic and which comprises a knockout of a polynucleotide according to claim 33.

46. A method for preparing a puree, juice or coulis of a fruit of a plant which comprises the step of using a fruit of a plant according to claim 39 for preparing said puree, juice or coulis.

47. A method for preparing an extract of a plant or plant part which comprises the step of using a plant or plant part of a plant according to claim 39 for preparing said extract.

48. The method of claim 47 wherein the extract is prepared without using organic solvents.

49. A method for preparing canned peeled tomatoes, tomato juice, tomato paste or tomato ketchup which comprises the step of using a tomato fruit of a tomato plant genetically engineered to not express at least partially a GDSL-lipase polypeptide according to claim 23 for preparing said canned peeled tomatoes, tomato juice, tomato paste or tomato ketchup.

50. A method for obtaining a plant mutant carrying at least a point mutation which at least partially suppresses the expression of a plant GDSL-lipase polypeptide according to claim 1, said method comprising the following steps:
(a) mutagenesis treatment of seeds of the plant with a chemical mutagen;
(b) self-fertilization of the resulting M1 plants to generate M2 plants;
(c) extraction of DNA samples of the M2 plants for mutational screening;
(d) pooling and arraying of the DNA samples in microtiter plates, wherein the pools are amplified using primers specific for a polynucleotide encoding the plant GDSL-lipase polypeptide;
(e) incubation of amplification products of step (d) with the CEL I or ENDO 1 endonuclease to generate cleavage products; and
(f) electrophoresis of cleavage products to identify mutations in the polynucleotide encoding the polypeptide to thereby identify a plant mutant carrying a point mutation in the GDSL-lipase polypeptide.

51. A method for preparing a puree, juice or coulis of a fruit of a plant which comprises the step of using a fruit of a plant mutant obtainable by the method of claim 50 for preparing said puree, juice or coulis.

52. A method for preparing an extract of a plant or plant part which comprises the step of using a mutant plant or part thereof obtainable by the method of claim 50 for preparing said extract.

53. The method of claim 52 wherein the extract is prepared without using organic solvents.

54. A method for preparing canned peeled tomatoes, tomato juice, tomato paste or tomato ketchup which comprises the step of using a tomato fruit carrying at least a point mutation obtainable by the method according to claim 50 for preparing said canned peeled tomatoes, tomato juice, tomato paste or tomato ketchup.


SEQUENCE LISTING (50 sequences)

SEQ ID NO: 1 - protein, 9 aa, Artificial
  Bloc1
XGDSLXDXG

SEQ ID NO: 2 - protein, 12 aa, Artificial
  Bloc2
TGRXXXGXXXXD

SEQ ID NO: 3 - protein, 4 aa, Artificial
  Bloc3
GXXX

SEQ ID NO: 4 - protein, 18 aa, Artificial
  Bloc4
XGAXRXXVXXXXPXGCXP

SEQ ID NO: 5 - protein, 16 aa, Artificial
  Bloc5
CXXXXXXXXWDXXHXX

SEQ ID NO: 6 - protein, 362 aa, Solanum lycopersicum
MATPTIILSFLLIFGVAICQSEARAFFVFGDSLVDSGNNNYLATTARADSPPYGIDYPTRRATGRFSNGYNIPDIISQQIGSSESPLPYLDPALTGQRLLVGANFASAGIGILNDTGIQFINIIRMPQQLAYFRQYQSRVSGLIGEANTQRLVNQALVLMTLGGNDFVNNYYLVPNSARSRQFSIQDYVPYLIREYRKILMNVYNLGARRVIVTGTGPLGCVPAELAQRSRNGECSPELQRAAGLFNPQLTQMLQGLNSELGSDVFIAANTQQMHTNFITNPQAYGFITSKVACCGQGPYNGLGLCTPLSNLCPNRDVYAFWDPFHPSERANKIIVQQIMSGTTELMNPMNLSTILAMDSHA

SEQ ID NO: 7 - protein, 369 aa, Solanum lycopersicum
MATFSLTKVSLSVLLVSLFFFSPSSAQTKCNVNSVYQLGDSLADAGNVIRTPGASIIFRADRSPYGETFFRKPTGRFSNGRVITDFISQSFKLPFLNAYLDRTASFSQGVNFAVAGATALDTSFWTARNIRLPTWNTPLSNQLTWFKSHLKSTCGSKCAENLKNSIVIMGEWGGNDYYNCFFQRKQISEVRSYVPFVVAGIMRGIKEVIQLGATRVLVPSIYPLGCLPLYLTSFPDNNTSGYDQLGCLKSFNDFASYHNRFVVRAMANLQRQFPNVSIVYGDFYGSILSLIRNPSSFGFNQNTLLSACCGTGGTHNFNFRTVCGGAGINACSNPAKYVHWDGIHLTDEAHRRISEVLVRDMLAKFNCVV

SEQ ID NO: 8 - protein, 380 aa, Solanum lycopersicum
MAFTIRVAVLHLLILEITIISSLFLQANAQELLIKLEEPKLKKCGINRIYQFGDSLSDTGNCLRESYCGAHTGCRFPPYGMNFYQKEITGRCSNGLLMVDFMALECGLPLLNPSLEENANFSQGSNFAVAGATALSVEQLAERNISMSYTNSTLTVQLHWMSSHFNSICSTDCPKYLEKSLFLVGEIGGDELAYGFIQGKTIQELRTMVPDIVHAIIRGVTRVISFGATRIVVPGNIPAGCNPVILTIFGTTYDEYGCVKEWNNFMMYYNNHLKRGVYMLKREYPNISISYGDYYNAYLWLRQNVVALGFDKNSVLKACCGIGGEYNYRADMRCGNPGVKACADPNSYISWDGIHLTQRAYSWLTKWLIDDIIPQLNCLV

SEQ ID NO: 9 - protein, 9 aa, Solanum lycopersicum
FGDSLVDSG

SEQ ID NO: 10 - protein, 12 aa, Artificial
  Bloc2
TGRXSNGXXXXD

SEQ ID NO: 11 - protein, 7 aa, Artificial
  Bloc3
GGNDXXN

SEQ ID NO: 12 - protein, 18 aa, Artificial
  Bloc4
LGAXRVXVXXXXPLGCXP

SEQ ID NO: 13 - protein, 20 aa, Artificial
  Bloc5
LCPNRXXYAFWDXFHPXEXA

SEQ ID NO: 14 - protein, 12 aa, Solanum lycopersicum
  misc_feature (8)..(8): X is any amino acid
TGRFSNGXNIXD

SEQ ID NO: 15 - protein, 13 aa, Artificial
  Bloc3
LVLXXLGGNDXXN

SEQ ID NO: 16 - protein, 16 aa, Solanum lycopersicum
  misc_feature (5)..(5): X is any amino acid
LGARXVXVTGTGPLGC

SEQ ID NO: 17 - protein, 20 aa, Artificial
  Bloc3
LVNXALVLXFXGGNDFVNNY

SEQ ID NO: 18 - protein, 3 aa, Solanum lycopersicum
PYG

SEQ ID NO: 19 - protein, 12 aa, Artificial
  Bloc between bloc2 and bloc3
GXNXAXAXXXXL

SEQ ID NO: 20 - protein, 12 aa, Artificial
  Bloc between bloc2 and bloc3
GXNFASAGIGIL

SEQ ID NO: 21 - protein, 12 aa, Solanum lycopersicum
  misc_feature (2)..(2): X is any amino acid
GXNFAVAGATAL

SEQ ID NO: 22 - protein, 6 aa, Artificial
  Bloc between bloc 4 and bloc5
ACCGXG

SEQ ID NO: 23 - protein, 8 aa, Solanum lycopersicum
  misc_feature (4)..(4): X is any amino acid
RADXPPYG

SEQ ID NO: 24 - DNA, 1432 bp, Solanum lycopersicum
ttggcggccaatcggccgaattgaaatgcacaaaccaatttataatatttttgtaacaacacacttttttttattttttttgccaaaatggccacacctactattattttgagcttcttgttgatttttggagtggctatttgtcaaagtgaagctagggcattttttgtgtttggtgattcacttgttgatagtggaaataataattatttggctactactgcaagggctgattcaccaccttatggtattgattatccaacacgtagagcaactggtcgtttctctaatggctacaacattcctgacattatcagtcaacaaattggttcatcagagtcaccactaccttacttagatccagctcttactggacaaagacttcttgttggtgctaactttgcatctgctggaattggaatactaaatgacactggaatccaatttattaatattattcgaatgccacaacaattggcttattttagacaatatcaaagtagagtaagtggccttattggtgaagcaaatactcaaagacttgtaaatcaagctcttgttcttatgactcttggaggcaatgattttgtcaacaactattatcttgtgcccaattctgcgcgatcacgccaattttctattcaagattatgtcccttatttgataagagaatatcgtaaaatcttgatgaatgtgtataatcttggagctcgtcgtgtaattgtaactggaactggaccgttaggttgtgttccagcagaactagctcaacgtagcaggaacggggaatgttcacccgagttgcaacgagctgcaggcctgtttaacccccagcttacgcaaatgttgcaggggttaaatagtgaactaggcagcgatgtttttattgctgcaaatacacaacaaatgcatacgaatttcattactaatccacaagcatatggatttataacatcaaaggtagcatgttgtggacaaggaccatataacggtcttggtctatgtacaccgctctctaatttgtgcccgaatagagatgtttacgcgttttgggacccgttccatccatctgagagggcaaataagatcattgtgcagcaaatcatgtctggtacaacggagcttatgaatccaatgaatctcagtacgattctggctatggattcacatgcataagacatatctaagatatctggaatctgattcacttgtaccttttttgttgctaatttttggctataaataagatgtatgcaacacttcatgttgtgctacttttaaatttacaaaaaagtttggttgtgtgctatgtttttattcacataattcagtaatctaatttgtagggtggagtgtgatattgttgaagatgtaaaccaaagtgtttttattaatttatatagtaatatattttcagtgtttaaaaaaaaaa

SEQ ID NO: 25 - DNA, 1110 bp, Solanum lycopersicum
atggcgactttttctcttaccaaagtctccttatccgttttactcgtctctttattcttcttttctccttcatctgctcaaactaaatgcaacgttaactccgtttaccaactcggcgactccctcgccgacgccggaaatgtcatccgtactcccggcgcttccatcatcttccgcgccgaccgttccccttacggcgaaactttcttcagaaaacccaccggccgcttctccaacggccgcgttatcaccgatttcatttctcaatcattcaaacttccattcctcaacgcttacttagacagaaccgcatcgttcagtcaaggagtaaacttcgccgtcgccggagctaccgccctcgatacatccttctggactgcacgtaacatccgtttacccacctggaacacaccgctttcaaatcaattaacttggttcaaatcgcatctgaaatcgacttgtggatcgaaatgtgcagagaatttgaaaaactcgatcgtaataatgggggaatggggaggaaacgattactacaattgtttcttccagagaaagcaaatctccgaagtcagaagttacgttccgtttgttgttgcagggataatgagaggcatcaaagaggtgattcagctgggagcaacacgagtgttggttccgagcatttaccctctcggatgccttccattatacctcacatccttccctgataacaatacaagtggttacgatcaattaggatgcctgaaaagcttcaatgatttcgcttcctatcacaatcgattcgtggttagagctatggccaatctacaacgtcaatttccaaatgttagtattgtttatggtgatttttatggatcaattttgtcacttattcgtaatccgtcttcgtttgggtttaatcaaaatacattgctgagtgcatgttgtggaactggaggaacacataatttcaacttcaggactgtatgtggaggagctgggattaatgcttgttcgaatccagcaaagtatgttcattgggacggaattcatttgacagatgaagctcatcgtcgtatttctgaagttctggttagagatatgttagccaaattcaactgcgtcgtttaa

SEQ ID NO: 26 - DNA, 1818 bp, Solanum lycopersicum
aggattcggcacgagggcattcacaataagagttgcggtgcttcatctgttgatactagaaataacaataataagttcgttgtttcttcaagctaacgcacaagaattattaataaagctcgaagaaccaaaattgaagaaatgcggaattaatagaatttatcagtttggtgattcactttccgataccggaaattgcctgagagagagctattgtggagctcatactgggtgtagattccctccttatggaatgaatttttaccagaaagaaataacggggcgttgttctaatggattgctcatggttgatttcatggcgctggaatgtggtcttcctctcctaaatccgtccttagaagaaaatgcaaattttagtcagggttcgaattttgcagtagcaggggctacggctttatcagttgaacaacttgcagaaaggaacatttctatgtcttatacaaatagtacattaactgtgcagcttcattggatgtcttctcatttcaattccatttgctctacggattgcccaaaatatttggaaaagtcacttttcctagttggagaaataggaggagatgaacttgcgtatgggtttatccaaggtaaaaccatacaagagttgcgaacaatggtgcctgacattgttcatgccattattcgcggtgttacatggcattcacaataagagttgcggtgcttcatctgttgatactagaaataacaataataagttcgttgtttcttcaagctaacgcacaagaattattaataaagctcgaagaaccaaaattgaagaaatgcggaattaatagaatttatcagtttggtgattcactttccgataccggaaattgcctgagagagagctattgtggagctcatactgggtgtagattccctccttatggaatgaatttttaccagaaagaaataacggggcgttgttctaatggattgctcatggttgatttcatggcgctggaatgtggtcttcctctcctaaatccgtccttagaagaaaatgcaaattttagtcagggttcgaattttgcagtagcaggggctacggctttatcagttgaacaacttgcagaaaggaacatttctatgtcttatacaaatagtacattaactgtgcagcttcattggatgtcttctcatttcaattccatttgctctacggattgcccaaaatatttggaaaagtcacttttcctagttggagaaataggaggagatgaacttgcgtatgggtttatccaaggtaaaaccatacaagagttgcgaacaatggtgcctgacattgttcatgccattattcgcggtgttacaagagttataagttttggagctactcgaattgtagttccaggtaatattccagctggttgtaacccggttatcctgacgatatttggtaccacatacgatgagtatggttgcgtcaaagagtggaacaattttatgatgtattacaacaatcatttgaaacgaggagtttacatgctcaagagagaatatccaaacatttcaatcagttacggcgactactacaacgcttatctgtggcttcgacaaaatgttgttgctcttggattcgacaaaaattctgtactgaaagcatgttgtggaataggtggagagtacaattatagagcagatatgagatgtggtaatccaggagtcaaagcgtgtgcagaccccaatagttacataagttgggatggaattcatttaacacaaagggcatacagttggttaacaaaatggctaatcgatgacataataccacaattaaactgccttgtttga

SEQ ID NO: 27 - DNA, 20 bp, artificial
  Primer GDSL1 3′
taaagcttatgcatgtgaat

SEQ ID NO: 28 - DNA, 31 bp, artificial
  GDSL1 5′
atccatggggcaaagtgaagctagggcattt

SEQ ID NO: 29 - protein, 16 aa, Solanum laciniatum
RAFFVFGDSLVDSGNN

SEQ ID NO: 30 - protein, 8 aa, Solanum lycopersicum
GGNDFVNN

SEQ ID NO: 31 - DNA, 32 bp, Artificial
  GDSL1s1 primer
aaaaagcaggctccaaaatggccacacctact

SEQ ID NO: 32 - DNA, 35 bp, Artificial
  GDSL1s2 primer
aaaaagcaggcttttttgttgctaatttttgccta

SEQ ID NO: 33 - DNA, 33 bp, Artificial
  GDSL1as3 primer
agaaagctgggttgacaaaaatcattgcctcca

SEQ ID NO: 34 - DNA, 36 bp, Artificial
  GDSL1as4 primer
agaaagctgggtcaacaatatccacactccacccta

SEQ ID NO: 35 - DNA, 31 bp, Artificial
  attB1 primer
ggggacaagtttgtacaaaaaagcaggctnn

SEQ ID NO: 36 - DNA, 31 bp, Artificial
  attB2 primer
ggggaccactttgtacaagaaagctgggtnn

SEQ ID NO: 37 - DNA, 24 bp, Artificial
  gdsl1 forward primer
gcaacacttcatgttgtgctactt

SEQ ID NO: 38 - DNA, 24 bp, Artificial
  gdsl1 reverse primer
caatatcacactccaccctacaaa

SEQ ID NO: 39 - DNA, 22 bp, Artificial
  gdsl2 forward primer
gccaaattcaactgcgtcgttt

SEQ ID NO: 40 - DNA, 22 bp, Artificial
  gdsl2 reverse primer
tctccttcgctacaacgtggaa

SEQ ID NO: 41 - DNA, 20 bp, Artificial
  gdsl3 forward primer
gggtggccaattacaaaatg

SEQ ID NO: 42 - DNA, 25 bp, Artificial
  gdsl3 reverse primer
actcacacacatcacataaagatgc

SEQ ID NO: 43 - DNA, 20 bp, Artificial
  b-tubulin forward primer
aacctctcgtggatcacagc

SEQ ID NO: 44 - DNA, 21 bp, Artificial
  b-tubulin reverse primer
ggcagaagctgtcaggtaacg

SEQ ID NO: 45 - DNA, 21 bp, Artificial
  EiF4a forward primer
agtggacgatttggaaggaag

SEQ ID NO: 46 - DNA, 20 bp, Artificial
  EiF4a reverse primer
gctcctcgattacgacgttg

SEQ ID NO: 47 - DNA, 21 bp, Artificial
  actin forward primer
ggactctggtgatggtgttag

SEQ ID NO: 48 - DNA, 19 bp, Artificial
  actin reverse primer
ccgttcagcagtagtggtg

SEQ ID NO: 49 - DNA, 510 bp, Artificial
  RNAi GDSL1
ccaaaatggccacacctactattattttgagcttcttgttgatttttggagtggctatttgtcaaagtgaagctagggcattttttgtgtttggtgattcacttgttgatagtggaaataataattatttggctactactgcaagggctgattcaccaccttatggtattgattatccaacacgtagagcaactggtcgtttctctaatggctacaacattcctgacattatcagtcaacaaattggttcatcagagtcaccactaccttacttagatccagctcttactggacaaagacttcttgttggtgctaactttgcatctgctggaattggaatactaaatgacactggaatccaatttattaatattattcgaatgccacaacaattggcttattttagacaatatcaaagtagagtaagtggccttattggtgaagcaaatactcaaagacttgtaaatcaagctcttgttcttatgactcttggaggcaatgattttgtca

SEQ ID NO: 50 - DNA, 148 bp, Artificial
  RNAi GDSL1
ttttttgttgctaatttttggctataaataagatgtatgcaacacttcatgttgtgctacttttaaatttacaaaaaagtttggttgtgtgctatgtttttattcacataattcagtaatctaatttgtagggtggagtgtgatattg